SEQUENCE LISTING (4 sequences)

SEQ ID NO: 1 - protein, 15 aa, Artificial
  Synthetic peptide
REVQSKIGYGRQIIS

SEQ ID NO: 2 - protein, 24 aa, Artificial
  Synthetic peptide
ELMTGELPYSHINNRDQIIFMVGR

SEQ ID NO: 3 - protein, 21 aa, Artificial
  Synthetic peptide
TGSLPYSHIGSRDQIIFMVGR

SEQ ID NO: 4 - protein, 18 aa, Artificial
  Synthetic peptide
GIVLYELMTGELPYSHIN

(19) United States
(12) Patent Application Publication
(10) Pub. No.: US 2015/0126405 A1
(43) Pub. Date: May 7, 2015

(54) POLYPEPTIDE MARKERS FOR THE EARLY RECOGNITION OF THE REJECTION OF TRANSPLANTED KIDNEYS

(71) Applicant: MOSAIQUES DIAGNOSTICS AND THERAPEUTICS AG, HANNOVER (DE)

(72) Inventors: Harald Mischak, Sehnde (DE); Stefan Wittke, Hannover (DE)

(21) Appl. No.: 14/554,368

(22) Filed: Nov. 26, 2014

Related U.S. Application Data
(63) Continuation of application No. 11/922,550, filed on May 13, 2008.

(30) Foreign Application Priority Data
Jun. 29, 2005 (EP) 05105822.0
Dec. 8, 2005 (DE) 102005058514.0
Dec. 22, 2005 (EP) 05112767.8

(51) Int. Cl. G01N 33/68

(52) U.S. Cl. CPC G01N 33/6893; G01N 33/6848; G01N 2800/245
USPC 506/12

(57) ABSTRACT

A method for recognizing rejection after a kidney transplantation (NTx), comprising the step of determining the presence or absence of at least one polypeptide marker in a sample, wherein said polypeptide marker is selected from markers 1 to 242 (frequency markers), or determining the amplitude of at least one polypeptide marker selected from markers 243 to 767 (amplitude markers), which are characterized by the values for the molecular masses and migration times (CE time).

### CROSS REFERENCE TO RELATED APPLICATIONS

This application is a continuation of and claims priority to U.S. patent application Ser. No. 11/922,550, filed May 13, 2008, which claims priority to PCT/EP2006/063365, filed Jun. 6, 2006, which, in turn, claims priority to European application number EP 05105822.0, filed Jun. 29, 2005, German application number DE 1002005058514.0, filed Dec. 8, 2005 and European application number EP 05112767.8, filed Dec. 22, 2005, the contents of which are all incorporated by reference herein in their entireties.

The present invention relates to the use of the presence or absence of one or more peptide markers in a sample from a subject for the early recognition of the rejection of transplanted kidneys, and to a method for the early recognition of the rejection of transplanted kidneys, wherein the presence or absence of the peptide marker or markers is indicative of the existence of rejection after kidney transplantation (NTx).

Kidney transplantation is the most frequently performed organ transplantation in Germany. Nevertheless, the waiting time for a new kidney is about 6-8 years. The goal of the therapy after kidney transplantation (NTx) has been performed is maintenance of functionality over as long a period as possible and prevention of rejection of the transplant. On average, 10-15% of the organs are lost after NTx due to various complications. Therefore, recognition of beginning rejection in due time is extraordinarily important because immediate medicinal intervention is necessary to preserve the transplant.

Surprisingly, it has now been found that particular peptide markers in a sample from a subject can be used for recognizing the rejection of transplanted kidneys.

Consequently, the present invention relates to the use of the presence or absence and amplitude of at least one polypeptide marker in a sample from a subject for recognizing the rejection of transplanted kidneys, wherein said polypeptide marker is selected from polypeptide marker Nos. 1 to 767 as characterized by the molecular masses and migration times as stated in Table 1.

TABLE 1
Polypeptide markers for recognizing the rejection
of transplanted kidneys and their molecular masses
(in Da) and migration times (CE time in minutes):

| Number | Mass | CE time |
|---|---|---|
| 1 | 2078.9 | 36.2 |
| 2 | 1168.6 | 40.8 |
| 3 | 2121.1 | 38 |
| 4 | 3359.6 | 48.1 |
| 5 | 1707.8 | 37.7 |
| 6 | 2815.9 | 36 |
| 7 | 3516.1 | 30 |
| 8 | 1697.9 | 34.7 |
| 9 | 2838.9 | 37.8 |
| 10 | 3482.1 | 31.4 |
| 11 | 8052.3 | 27.8 |
| 12 | 1027.6 | 35.8 |
| 13 | 1031.6 | 37.5 |
| 14 | 3240.2 | 34 |
| 15 | 1811 | 42.3 |
| 16 | 1016.46 | 33.49 |
| 17 | 1026.47 | 35.31 |
| 18 | 1032.45 | 33.66 |
| 19 | 1059.69 | 33.55 |
| 20 | 1065.55 | 25.50 |
| 21 | 1078.52 | 27.75 |
| 22 | 1100.46 | 24.80 |
| 23 | 1100.58 | 21.83 |
| 24 | 1110.43 | 33.78 |
| 25 | 1112.56 | 23.99 |
| 26 | 1125.55 | 24.64 |
| 27 | 1132.61 | 23.70 |
| 28 | 1137.58 | 26.41 |
| 29 | 1137.67 | 28.70 |
| 30 | 1141.57 | 24.65 |
| 31 | 1150.61 | 22.43 |
| 32 | 1159.64 | 26.05 |
| 33 | 1162.59 | 20.21 |
| 34 | 1171.55 | 29.24 |
| 35 | 1178.6 | 26.84 |
| 36 | 1180.56 | 35.61 |
| 37 | 1185.59 | 26.38 |
| 38 | 1195.57 | 37.51 |
| 39 | 1197.56 | 21.09 |
| 40 | 1202.53 | 35.63 |
| 41 | 1211.69 | 21.38 |
| 42 | 1212.68 | 21.43 |
| 43 | 1217.6 | 39.21 |
| 44 | 1226.58 | 21.02 |
| 45 | 1232.59 | 24.37 |
| 46 | 1238.55 | 36.01 |
| 47 | 1239.54 | 35.39 |
| 48 | 1240.61 | 23.68 |
| 49 | 1245.81 | 33.86 |
| 50 | 1255.53 | 35.72 |
| 51 | 1263.61 | 19.74 |
| 52 | 1273.65 | 27.06 |
| 53 | 1276.75 | 19.89 |
| 54 | 1279.69 | 19.68 |
| 55 | 1285.63 | 28.14 |
| 56 | 1287.65 | 21.85 |
| 57 | 1297.77 | 21.90 |
| 58 | 1324.63 | 24.80 |
| 59 | 1348.68 | 20.23 |
| 60 | 1351.69 | 38.75 |
| 61 | 1357.67 | 22.44 |
| 62 | 1358.66 | 26.84 |
| 63 | 1379.68 | 28.05 |
| 64 | 1384.71 | 38.77 |
| 65 | 1400.72 | 22.44 |
| 66 | 1404.91 | 34.89 |
| 67 | 1405.71 | 20.16 |
| 68 | 1422.81 | 34.49 |
| 69 | 1426.71 | 19.92 |
| 70 | 1434.76 | 22.56 |
| 71 | 1437.72 | 30.40 |
| 72 | 1441.67 | 19.94 |
| 73 | 1441.73 | 28.87 |
| 74 | 1443.72 | 20.46 |
| 75 | 1446.68 | 19.74 |
| 76 | 1451.69 | 36.04 |
| 77 | 1456.71 | 29.28 |
| 78 | 1457.68 | 21.95 |
| 79 | 1462.67 | 39.43 |
| 80 | 1462.75 | 22.81 |
| 81 | 1466.72 | 28.51 |
| 82 | 1494.72 | 30.40 |
| 83 | 1513.7 | 29.29 |
| 84 | 1539.92 | 33.44 |
| 85 | 1545.75 | 29.24 |
| 86 | 1554.72 | 28.64 |
| 87 | 1561.77 | 29.45 |
| 88 | 1583.76 | 30.11 |
| 89 | 1584.77 | 29.72 |
| 90 | 1587.8 | 19.91 |
| 91 | 1621.78 | 30.05 |
| 92 | 1633.73 | 31.93 |
| 93 | 1635.72 | 37.66 |
| 94 | 1652.83 | 22.64 |
| 95 | 1658.8 | 29.91 |
| 96 | 1666.84 | 30.65 |
| 97 | 1674.82 | 23.24 |
| 98 | 1678.83 | 30.84 |
| 99 | 1688.76 | 20.22 |
| 100 | 1689.86 | 27.59 |
| 101 | 1692.86 | 32.09 |
| 102 | 1711.72 | 22.68 |
| 103 | 1713.83 | 30.77 |
| 104 | 1732.84 | 28.21 |
| 105 | 1734.87 | 23.61 |
| 106 | 1741.81 | 30.21 |
| 107 | 1749.85 | 20.00 |
| 108 | 1749.88 | 30.54 |
| 109 | 1764.86 | 29.88 |
| 110 | 1768.9 | 20.77 |
| 111 | 1783.86 | 39.97 |
| 112 | 1784.88 | 39.94 |
| 113 | 1809.91 | 20.98 |
| 114 | 1812.9 | 39.98 |
| 115 | 1813.78 | 31.87 |
| 116 | 1814.84 | 30.49 |
| 117 | 1822.81 | 30.87 |
| 118 | 1825.87 | 31.80 |
| 119 | 1832.92 | 31.91 |
| 120 | 1837.88 | 30.54 |
| 121 | 1852.85 | 31.25 |
| 122 | 1882.9 | 22.09 |
| 123 | 1885.95 | 21.25 |
| 124 | 1889.97 | 19.38 |
| 125 | 1930.94 | 20.67 |
| 126 | 1930.97 | 31.53 |
| 127 | 1938.96 | 21.39 |
| 128 | 1951.93 | 32.08 |
| 129 | 2059.02 | 23.12 |
| 130 | 2066.98 | 25.11 |
| 131 | 2096.91 | 20.23 |
| 132 | 2119.07 | 25.45 |
| 133 | 2124.94 | 20.39 |
| 134 | 2155.06 | 27.54 |
| 135 | 2158.12 | 22.08 |
| 136 | 2178.1 | 34.07 |
| 137 | 2189.08 | 27.17 |
| 138 | 2192.07 | 22.37 |
| 139 | 2233.14 | 20.52 |
| 140 | 2274.11 | 33.47 |
| 141 | 2286.06 | 25.98 |
| 142 | 2289.47 | 33.56 |
| 143 | 2305.65 | 34.65 |
| 144 | 2308.1 | 27.32 |
| 145 | 2313.11 | 33.57 |
| 146 | 2320.16 | 20.73 |
| 147 | 2349.14 | 27.36 |
| 148 | 2353.83 | 27.12 |
| 149 | 2356.74 | 35.53 |
| 150 | 2420.11 | 34.09 |
| 151 | 2459.17 | 34.05 |
| 152 | 2471.25 | 34.69 |
| 153 | 2474.24 | 19.77 |
| 154 | 2483.28 | 29.20 |
| 155 | 2487.18 | 25.95 |
| 156 | 2501.84 | 20.05 |
| 157 | 2524.62 | 19.81 |
| 158 | 2544.23 | 26.11 |
| 159 | 2564.77 | 27.19 |
| 160 | 2600.93 | 34.67 |
| 161 | 2658.1 | 21.45 |
| 162 | 2680.08 | 27.98 |
| 163 | 2686.97 | 29.06 |
| 164 | 2695.41 | 27.37 |
| 165 | 2713.31 | 29.13 |
| 166 | 2726.37 | 42.97 |
| 167 | 2728.65 | 29.05 |
| 168 | 2825.34 | 26.86 |
| 169 | 2854.27 | 21.54 |
| 170 | 2875.09 | 29.21 |
| 171 | 2891.43 | 29.40 |
| 172 | 2940.95 | 29.07 |
| 173 | 3001.26 | 37.28 |
| 174 | 3002.23 | 23.80 |
| 175 | 3005.84 | 24.48 |
| 176 | 3021.47 | 35.35 |
| 177 | 3023.05 | 37.20 |
| 178 | 3031.39 | 35.93 |
| 179 | 3046.47 | 20.64 |
| 180 | 3047.99 | 29.45 |
| 181 | 3048.04 | 35.82 |
| 182 | 3054.49 | 22.03 |
| 183 | 3108.55 | 31.22 |
| 184 | 3124.19 | 19.82 |
| 185 | 3182 | 30.72 |
| 186 | 3185.29 | 30.71 |
| 187 | 3238.04 | 29.05 |
| 188 | 3264.75 | 30.51 |
| 189 | 3272.4 | 36.47 |
| 190 | 3276.21 | 33.32 |
| 191 | 3344.47 | 20.95 |
| 192 | 3356.59 | 22.08 |
| 193 | 3378.05 | 38.81 |
| 194 | 3378.33 | 31.44 |
| 195 | 3400.13 | 31.50 |
| 196 | 3479.24 | 33.46 |
| 197 | 3515.51 | 23.23 |
| 198 | 3515.81 | 20.63 |
| 199 | 3536.54 | 20.35 |
| 200 | 3552.27 | 20.67 |
| 201 | 3576.5 | 21.47 |
| 202 | 3612.65 | 23.03 |
| 203 | 3617.74 | 26.97 |
| 204 | 3633.69 | 26.99 |
| 205 | 3657.59 | 23.19 |
| 206 | 3735.52 | 27.10 |
| 207 | 3798.16 | 20.18 |
| 208 | 3802.27 | 37.66 |
| 209 | 3805.59 | 27.63 |
| 210 | 3870.85 | 33.39 |
| 211 | 3890.96 | 27.27 |
| 212 | 3927.78 | 19.76 |
| 213 | 3968.58 | 25.01 |
| 214 | 3988.76 | 27.65 |
| 215 | 3991.47 | 34.46 |
| 216 | 4008.93 | 23.43 |
| 217 | 4015.99 | 28.02 |
| 218 | 4022.38 | 32.97 |
| 219 | 4041.27 | 20.42 |
| 220 | 4062.7 | 33.07 |
| 221 | 4101.34 | 28.51 |
| 222 | 4122.34 | 20.11 |
| 223 | 4160.09 | 28.82 |
| 224 | 4190.81 | 20.55 |
| 225 | 4236.87 | 24.02 |
| 226 | 4387.08 | 21.01 |
| 227 | 4584.13 | 24.18 |
| 228 | 4601.03 | 23.62 |
| 229 | 4624.83 | 19.89 |
| 230 | 4777.58 | 21.17 |
| 231 | 5000.89 | 30.20 |
| 232 | 5042.57 | 20.20 |
| 233 | 5143.4 | 20.17 |
| 234 | 5175.89 | 21.70 |
| 235 | 5340.29 | 25.10 |
| 236 | 5411.46 | 20.13 |
| 237 | 5831.21 | 23.80 |
| 238 | 6074.38 | 23.25 |
| 239 | 6629.99 | 27.15 |
| 240 | 7907.23 | 19.59 |
| 241 | 9972.75 | 19.67 |
| 242 | 11777.18 | 19.55 |
| 243 | 1006.52 | 24.04 |
| 244 | 1008.55 | 22.65 |
| 245 | 1025.51 | 25.45 |
| 246 | 1032.49 | 25.94 |
| 247 | 1046.55 | 25.35 |
| 248 | 1050.52 | 27.03 |
| 249 | 1051.43 | 25.13 |
| 250 | 1057.68 | 32.58 |
| 251 | 1059.7 | 32.04 |
| 252 | 1064.55 | 21.14 |
| 253 | 1068.5 | 29.20 |
| 254 | 1068.51 | 26.96 |
| 255 | 1080.53 | 27.86 |
| 256 | 1081.66 | 20.79 |
| 257 | 1082.55 | 24.02 |
| 258 | 1082.56 | 21.30 |
| 259 | 1083.56 | 21.63 |
| 260 | 1084.6 | 21.29 |
| 261 | 1094.63 | 19.37 |
| 262 | 1095.34 | 37.65 |
| 263 | 1096.41 | 35.95 |
| 264 | 1096.65 | 20.83 |
| 265 | 1097.54 | 25.45 |
| 266 | 1112.6 | 21.11 |
| 267 | 1114.52 | 28.69 |
| 268 | 1127.54 | 25.47 |
| 269 | 1128.58 | 21.52 |
| 270 | 1134.63 | 23.61 |
| 271 | 1139.55 | 21.01 |
| 272 | 1142.6 | 24.66 |
| 273 | 1152.55 | 26.07 |
| 274 | 1160.47 | 37.93 |
| 275 | 1160.67 | 34.11 |
| 276 | 1162.64 | 37.21 |
| 277 | 1168.61 | 20.98 |
| 278 | 1170.64 | 21.67 |
| 279 | 1174.58 | 35.65 |
| 280 | 1190.68 | 33.65 |
| 281 | 1198.6 | 26.04 |
| 282 | 1200.59 | 24.26 |
| 283 | 1208.55 | 26.46 |
| 284 | 1210.43 | 36.48 |
| 285 | 1211.59 | 25.89 |
| 286 | 1216.59 | 24.25 |
| 287 | 1217.58 | 35.75 |
| 288 | 1220.64 | 22.05 |
| 289 | 1222.61 | 26.78 |
| 290 | 1250.69 | 20.39 |
| 291 | 1253.59 | 25.29 |
| 292 | 1260.61 | 21.81 |
| 293 | 1261.67 | 22.21 |
| 294 | 1265.64 | 27.10 |
| 295 | 1270.65 | 29.41 |
| 296 | 1283.62 | 27.30 |
| 297 | 1296.62 | 27.40 |
| 298 | 1296.65 | 19.38 |
| 299 | 1296.66 | 25.40 |
| 300 | 1299.85 | 33.33 |
| 301 | 1304.66 | 21.86 |
| 302 | 1306.64 | 27.49 |
| 303 | 1317.65 | 27.49 |
| 304 | 1317.87 | 34.06 |
| 305 | 1321.65 | 28.39 |
| 306 | 1322.66 | 27.46 |
| 307 | 1326.61 | 29.26 |
| 308 | 1353.71 | 25.66 |
| 309 | 1359.64 | 27.97 |
| 310 | 1360.7 | 22.39 |
| 311 | 1364.82 | 19.39 |
| 312 | 1367.7 | 38.85 |
| 313 | 1370.68 | 27.77 |
| 314 | 1375.67 | 23.29 |
| 315 | 1378.67 | 28.88 |
| 316 | 1381.68 | 27.39 |
| 317 | 1383.65 | 27.75 |
| 318 | 1388.7 | 27.91 |
| 319 | 1392.69 | 21.73 |
| 320 | 1401.68 | 28.02 |
| 321 | 1422.66 | 21.70 |
| 322 | 1424.72 | 39.25 |
| 323 | 1435.59 | 19.78 |
| 324 | 1438.73 | 21.91 |
| 325 | 1444.79 | 19.98 |
| 326 | 1463.75 | 19.48 |
| 327 | 1467.73 | 21.87 |
| 328 | 1474.74 | 22.43 |
| 329 | 1476.68 | 27.76 |
| 330 | 1482.73 | 22.47 |
| 331 | 1483.72 | 22.58 |
| 332 | 1484.74 | 23.51 |
| 333 | 1488.66 | 20.12 |
| 334 | 1492.73 | 28.98 |
| 335 | 1496.75 | 30.36 |
| 336 | 1499.93 | 21.20 |
| 337 | 1501.8 | 20.12 |
| 338 | 1512.75 | 26.68 |
| 339 | 1518.68 | 19.38 |
| 340 | 1522.78 | 22.76 |
| 341 | 1525.66 | 22.23 |
| 342 | 1526.76 | 23.65 |
| 343 | 1534.69 | 19.89 |
| 344 | 1535.75 | 29.20 |
| 345 | 1538.7 | 22.56 |
| 346 | 1560.68 | 21.98 |
| 347 | 1575.81 | 30.13 |
| 348 | 1576.82 | 19.52 |
| 349 | 1583.67 | 19.45 |
| 350 | 1592.77 | 22.15 |
| 351 | 1592.8 | 19.53 |
| 352 | 1593.78 | 22.50 |
| 353 | 1612.83 | 23.36 |
| 354 | 1617.79 | 23.21 |
| 355 | 1622.78 | 30.47 |
| 356 | 1622.79 | 26.82 |
| 357 | 1623.8 | 29.64 |
| 358 | 1624.61 | 37.75 |
| 359 | 1626.77 | 23.09 |
| 360 | 1635.85 | 40.40 |
| 361 | 1640.65 | 23.38 |
| 362 | 1648.8 | 30.11 |
| 363 | 1649.79 | 19.59 |
| 364 | 1652.8 | 20.07 |
| 365 | 1656.82 | 25.48 |
| 366 | 1673.81 | 29.65 |
| 367 | 1673.86 | 27.48 |
| 368 | 1690.88 | 19.77 |
| 369 | 1692.86 | 30.74 |
| 370 | 1694.82 | 23.44 |
| 371 | 1697.81 | 30.85 |
| 372 | 1698.65 | 37.60 |
| 373 | 1702.75 | 19.81 |
| 374 | 1703.9 | 33.64 |
| 375 | 1708.84 | 32.09 |
| 376 | 1708.85 | 30.44 |
| 377 | 1716.38 | 20.59 |
| 378 | 1716.84 | 28.03 |
| 379 | 1732.85 | 32.41 |
| 380 | 1732.85 | 30.25 |
| 381 | 1733.82 | 19.34 |
| 382 | 1734.8 | 20.24 |
| 383 | 1761.77 | 19.37 |
| 384 | 1764.76 | 20.02 |
| 385 | 1765.88 | 30.91 |
| 386 | 1765.96 | 19.81 |
| 387 | 1778.8 | 30.36 |
| 388 | 1793.01 | 21.18 |
| 389 | 1794.83 | 30.37 |
| 390 | 1798.85 | 31.81 |
| 391 | 1817.77 | 20.34 |
| 392 | 1829.04 | 21.22 |
| 393 | 1835.79 | 20.02 |
| 394 | 1841.96 | 24.38 |
| 395 | 1844.56 | 34.28 |
| 396 | 1859.9 | 24.38 |
| 397 | 1878.92 | 20.73 |
| 398 | 1916.84 | 20.42 |
| 399 | 1924.03 | 21.43 |
| 400 | 1927.99 | 19.53 |
| 401 | 1934.87 | 20.04 |
| 402 | 1936.96 | 32.25 |
| 403 | 1940.02 | 21.73 |
| 404 | 1955.03 | 25.27 |
| 405 | 1989.96 | 32.40 |
| 406 | 2004.02 | 24.62 |
| 407 | 2034.96 | 19.58 |
| 408 | 2049 | 22.85 |
| 409 | 2067.93 | 20.68 |
| 410 | 2082.01 | 33.67 |
| 411 | 2087.97 | 19.47 |
| 412 | 2090.77 | 19.74 |
| 413 | 2101.03 | 21.96 |
| 414 | 2103.95 | 19.43 |
| 415 | 2104.06 | 34.88 |
| 416 | 2129.06 | 27.03 |
| 417 | 2133.03 | 32.63 |
| 418 | 2148.1 | 25.54 |
| 419 | 2157.11 | 19.51 |
| 420 | 2159.04 | 34.88 |
| 421 | 2171.03 | 27.65 |
| 422 | 2188.03 | 39.81 |
| 423 | 2192 | 33.15 |
| 424 | 2195.06 | 20.15 |
| 425 | 2199.02 | 22.30 |
| 426 | 2203.19 | 21.91 |
| 427 | 2216.11 | 33.79 |
| 428 | 2217.69 | 33.71 |
| 429 | 2235.13 | 34.10 |
| 430 | 2243.09 | 26.28 |
| 431 | 2248.43 | 33.56 |
| 432 | 2248.8 | 22.49 |
| 433 | 2249.17 | 20.53 |
| 434 | 2249.2 | 26.27 |
| 435 | 2257.64 | 36.10 |
| 436 | 2261.13 | 27.19 |
| 437 | 2261.15 | 25.94 |
| 438 | 2269.11 | 39.33 |
| 439 | 2276.44 | 27.20 |
| 440 | 2279.62 | 23.39 |
| 441 | 2281.89 | 22.24 |
| 442 | 2282.11 | 26.27 |
| 443 | 2292.11 | 27.26 |
| 444 | 2302.36 | 26.12 |
| 445 | 2320.13 | 34.31 |
| 446 | 2321.61 | 22.12 |
| 447 | 2323.13 | 22.33 |
| 448 | 2337.69 | 22.59 |
| 449 | 2339.08 | 33.95 |
| 450 | 2356.24 | 19.54 |
| 451 | 2361.22 | 20.77 |
| 452 | 2404.11 | 20.27 |
| 453 | 2405.59 | 22.16 |
| 454 | 2427.26 | 19.59 |
| 455 | 2458.9 | 20.66 |
| 456 | 2467.42 | 27.92 |
| 457 | 2483.21 | 27.54 |
| 458 | 2507.21 | 22.76 |
| 459 | 2516.64 | 20.48 |
| 460 | 2551.24 | 34.63 |
| 461 | 2555.5 | 27.90 |
| 462 | 2564.64 | 23.25 |
| 463 | 2565.03 | 24.71 |
| 464 | 2567.15 | 34.72 |
| 465 | 2570.28 | 42.64 |
| 466 | 2578.55 | 27.44 |
| 467 | 2589.17 | 22.45 |
| 468 | 2607.78 | 26.93 |
| 469 | 2608.95 | 19.55 |
| 470 | 2616.02 | 28.35 |
| 471 | 2621.46 | 19.89 |
| 472 | 2622.48 | 26.80 |
| 473 | 2629.85 | 19.84 |
| 474 | 2647.25 | 23.43 |
| 475 | 2649.35 | 34.79 |
| 476 | 2659.35 | 19.81 |
| 477 | 2674.74 | 20.41 |
| 478 | 2694.21 | 32.20 |
| 479 | 2724.07 | 23.84 |
| 480 | 2743.25 | 19.70 |
| 481 | 2744.07 | 35.03 |
| 482 | 2758.36 | 28.94 |
| 483 | 2839.43 | 24.14 |
| 484 | 2910.93 | 21.67 |
| 485 | 2912.24 | 25.62 |
| 486 | 2917.54 | 28.99 |
| 487 | 2920.26 | 21.13 |
| 488 | 2925.27 | 28.61 |
| 489 | 2926.16 | 22.14 |
| 490 | 2946.45 | 34.96 |
| 491 | 2994.09 | 29.50 |
| 492 | 3003.89 | 20.02 |
| 493 | 3013.24 | 22.27 |
| 494 | 3035 | 42.12 |
| 495 | 3038.1 | 22.28 |
| 496 | 3039.44 | 24.60 |
| 497 | 3049.47 | 24.97 |
| 498 | 3058.02 | 30.20 |
| 499 | 3092.53 | 31.18 |
| 500 | 3141.11 | 20.08 |
| 501 | 3143.51 | 32.84 |
| 502 | 3149.56 | 31.20 |
| 503 | 3154.38 | 20.60 |
| 504 | 3165.49 | 31.25 |
| 505 | 3201.54 | 23.03 |
| 506 | 3207.65 | 24.70 |
| 507 | 3223.03 | 21.36 |
| 508 | 3224.99 | 25.39 |
| 509 | 3238.96 | 22.31 |
| 510 | 3252.33 | 29.04 |
| 511 | 3256.55 | 33.01 |
| 512 | 3261.75 | 19.47 |
| 513 | 3273.55 | 22.65 |
| 514 | 3288.18 | 36.47 |
| 515 | 3322.57 | 19.88 |
| 516 | 3334.2 | 30.93 |
| 517 | 3353.93 | 23.53 |
| 518 | 3359.67 | 31.84 |
| 519 | 3361.42 | 24.26 |
| 520 | 3368.53 | 21.34 |
| 521 | 3405.14 | 30.25 |
| 522 | 3416.42 | 31.94 |
| 523 | 3435.8 | 19.96 |
| 524 | 3441.51 | 34.32 |
| 525 | 3443.94 | 23.07 |
| 526 | 3448.69 | 32.05 |
| 527 | 3479.63 | 31.71 |
| 528 | 3482.5 | 21.77 |
| 529 | 3524.51 | 32.24 |
| 530 | 3543.73 | 30.01 |
| 531 | 3551.59 | 26.23 |
| 532 | 3582.72 | 19.48 |
| 533 | 3593.53 | 20.25 |
| 534 | 3608.71 | 28.79 |
| 535 | 3630.57 | 24.45 |
| 536 | 3642.74 | 19.86 |
| 537 | 3680.25 | 26.86 |
| 538 | 3694.69 | 20.19 |
| 539 | 3696.88 | 26.94 |
| 540 | 3702.39 | 32.39 |
| 541 | 3706.61 | 21.99 |
| 542 | 3718.81 | 32.39 |
| 543 | 3734.85 | 32.41 |
| 544 | 3754.66 | 37.16 |
| 545 | 3763.03 | 19.81 |
| 546 | 3828.64 | 33.79 |
| 547 | 3855.29 | 20.69 |
| 548 | 3862.83 | 25.16 |
| 549 | 3893.69 | 19.82 |
| 550 | 3954.09 | 19.95 |
| 551 | 3959.8 | 19.95 |
| 552 | 3986.76 | 20.66 |
| 553 | 3992.9 | 19.80 |
| 554 | 4012.41 | 20.81 |
| 555 | 4016.27 | 22.03 |
| 556 | 4016.85 | 19.82 |
| 557 | 4043.72 | 20.43 |
| 558 | 4059.96 | 20.44 |
| 559 | 4066.96 | 21.03 |
| 560 | 4113.8 | 24.58 |
| 561 | 4140.04 | 19.89 |
| 562 | 4169.63 | 35.26 |
| 563 | 4227.17 | 21.24 |
| 564 | 4229.09 | 29.08 |
| 565 | 4250 | 28.08 |
| 566 | 4251.98 | 28.66 |
| 567 | 4252.83 | 24.45 |
| 568 | 4265.76 | 25.70 |
| 569 | 4278.73 | 23.34 |
| 570 | 4288.98 | 25.94 |
| 571 | 4299.15 | 33.05 |
| 572 | 4322.21 | 27.00 |
| 573 | 4352.95 | 20.24 |
| 574 | 4369.06 | 20.25 |
| 575 | 4379.78 | 28.40 |
| 576 | 4382.08 | 28.00 |
| 577 | 4404.96 | 20.69 |
| 578 | 4410.01 | 20.05 |
| 579 | 4418.7 | 25.78 |
| 580 | 4457.48 | 22.96 |
| 581 | 4468.94 | 21.20 |
| 582 | 4510.28 | 26.44 |
| 583 | 4536.25 | 20.01 |
| 584 | 4557.06 | 20.10 |
| 585 | 4579.04 | 25.81 |
| 586 | 4675.96 | 20.59 |
| 587 | 4718.21 | 27.64 |
| 588 | 4758.98 | 29.87 |
| 589 | 4771.23 | 20.23 |
| 590 | 4787.34 | 20.28 |
| 591 | 4816.29 | 29.92 |
| 592 | 4890.88 | 26.48 |
| 593 | 4907.13 | 26.50 |
| 594 | 4960.51 | 20.59 |
| 595 | 4976.45 | 20.58 |
| 596 | 5000.2 | 26.36 |
| 597 | 5090.44 | 20.05 |
| 598 | 5106.57 | 20.06 |
| 599 | 5158.25 | 25.08 |
| 600 | 5221.6 | 20.24 |
| 601 | 5228.18 | 27.02 |
| 602 | 5382.32 | 19.50 |
| 603 | 5394.49 | 20.06 |
| 604 | 5427.38 | 22.98 |
| 605 | 5495.48 | 19.51 |
| 606 | 5527.56 | 27.58 |
| 607 | 5921.87 | 20.49 |
| 608 | 6783.04 | 26.61 |
| 609 | 7975.01 | 34.53 |
| 610 | 8020.22 | 19.60 |
| 611 | 8111.07 | 19.85 |
| 612 | 8559.57 | 19.59 |
| 613 | 8853.85 | 21.08 |
| 614 | 8933.94 | 22.57 |
| 615 | 10216.66 | 21.07 |
| 616 | 10330.33 | 21.57 |
| 617 | 10366.55 | 21.21 |
| 618 | 11058.18 | 21.88 |
| 619 | 11491.09 | 21.54 |
| 620 | 11967.96 | 20.50 |
| 621 | 12717.08 | 26.92 |
| 622 | 12879.25 | 26.95 |
| 623 | 13008.02 | 28.79 |
| 624 | 13170.16 | 29.03 |
| 625 | 13373.35 | 29.18 |
| 626 | 14111.27 | 21.97 |
| 627 | 15817.61 | 19.42 |
| 628 | 833.36 | 27.38 |
| 629 | 838.44 | 35.05 |
| 630 | 840.44 | 23.94 |
| 631 | 854.38 | 34.99 |
| 632 | 860.4 | 26.23 |
| 633 | 911.47 | 25.92 |
| 634 | 922.45 | 22 |
| 635 | 945.46 | 25.7 |
| 636 | 965.46 | 27.63 |
| 637 | 1035.5 | 21.12 |
| 638 | 1038.54 | 35.97 |
| 639 | 1040.41 | 35.22 |
| 640 | 1068.56 | 21.69 |
| 641 | 1100.55 | 36.99 |
| 642 | 1135.53 | 27.51 |
| 643 | 1174.75 | 33.53 |
| 644 | 1229.57 | 36.29 |
| 645 | 1235.61 | 26.73 |
| 646 | 1245.6 | 21.63 |
| 647 | 1246.61 | 21.82 |
| 648 | 1247.58 | 22.02 |
| 649 | 1252.67 | 21.57 |
| 650 | 1272.41 | 35.91 |
| 651 | 1296.64 | 26.52 |
| 652 | 1297.72 | 31.96 |
| 653 | 1302.7 | 21.77 |
| 654 | 1304.59 | 27.91 |
| 655 | 1310.65 | 20.93 |
| 656 | 1312.66 | 32.36 |
| 657 | 1328.64 | 26.87 |
| 658 | 1329.7 | 22.63 |
| 659 | 1333.47 | 36.13 |
| 660 | 1337.67 | 38.1 |
| 661 | 1386.73 | 35.78 |
| 662 | 1405.64 | 38.57 |
| 663 | 1406.69 | 28.26 |
| 664 | 1412.51 | 37.16 |
| 665 | 1412.73 | 28.47 |
| 666 | 1419.75 | 28.4 |
| 667 | 1426.7 | 22.54 |
| 668 | 1460.86 | 22.73 |
| 669 | 1491.8 | 39.86 |
| 670 | 1504.73 | 20.74 |
| 671 | 1521.75 | 30.38 |
| 672 | 1523.8 | 40.46 |
| 673 | 1523.89 | 33.7 |
| 674 | 1563.77 | 29.56 |
| 675 | 1576.74 | 46.02 |
| 676 | 1577.75 | 40.06 |
| 677 | 1588.77 | 30.2 |
| 678 | 1631.78 | 47.02 |
| 679 | 1640.75 | 28.28 |
| 680 | 1669.78 | 21.47 |
| 681 | 1708.85 | 31.08 |
| 682 | 1720.76 | 19.69 |
| 683 | 1731.88 | 39.02 |
| 684 | 1769.87 | 39.62 |
| 685 | 1796.85 | 30.86 |
| 686 | 1808.87 | 23.72 |
| 687 | 1852.96 | 25.14 |
| 688 | 1859.87 | 31.04 |
| 689 | 1863.95 | 44.05 |
| 690 | 1880.97 | 44.03 |
| 691 | 1891.65 | 54.28 |
| 692 | 1952.42 | 25.65 |
| 693 | 2035.01 | 25.44 |
| 694 | 2045.94 | 34.11 |
| 695 | 2168.71 | 35.13 |
| 696 | 2184.86 | 33.93 |
| 697 | 2205.06 | 30.71 |
| 698 | 2210.94 | 37.92 |
| 699 | 2212.06 | 33.53 |
| 700 | 2219.65 | 27.04 |
| 701 | 2228.47 | 32.96 |
| 702 | 2248.09 | 28.54 |
| 703 | 2249.66 | 34.06 |
| 704 | 2260.14 | 25.75 |
| 705 | 2292.21 | 21.29 |
| 706 | 2298.65 | 26.68 |
| 707 | 2318.29 | 26.28 |
| 708 | 2320.79 | 22.48 |
| 709 | 2384.98 | 20.97 |
| 710 | 2421.46 | 22.19 |
| 711 | 2423.4 | 21.03 |
| 712 | 2430.22 | 25.64 |
| 713 | 2529.25 | 27.68 |
| 714 | 2534.56 | 22.81 |
| 715 | 2579.8 | 27.46 |
| 716 | 2592.17 | 42.19 |
| 717 | 2632.57 | 34.44 |
| 718 | 2742.36 | 42.12 |
| 719 | 2752.54 | 19.92 |
| 720 | 2755.34 | 28.02 |
| 721 | 2767.29 | 21.59 |
| 722 | 2773.21 | 20.93 |
| 723 | 2802.82 | 36.21 |
| 724 | 2830.66 | 21.07 |
| 725 | 2834.44 | 22.26 |
| 726 | 2907.44 | 35.9 |
| 727 | 2936.45 | 20.74 |
| 728 | 2977.61 | 29.14 |
| 729 | 3015.84 | 35.9 |
| 730 | 3042.3 | 34.08 |
| 731 | 3048.09 | 29.51 |
| 732 | 3145.49 | 38.8 |
| 733 | 3148.42 | 24.17 |
| 734 | 3205.44 | 23.65 |
| 735 | 3242.43 | 22.81 |
| 736 | 3292.64 | 39.38 |
| 737 | 3298.48 | 36.06 |
| 738 | 3330.16 | 30.54 |
| 739 | 3343.38 | 31.82 |
| 740 | 3371.77 | 23 |
| 741 | 3400.07 | 42.03 |
| 742 | 3457.46 | 35.83 |
| 743 | 3465.5 | 26.98 |
| 744 | 3559.71 | 24.89 |
| 745 | 3596.51 | 21.57 |
| 746 | 3605.63 | 21.22 |
| 747 | 3612.53 | 22.99 |
| 748 | 3617.77 | 26.98 |
| 749 | 3633.71 | 27.02 |
| 750 | 3774.84 | 22.9 |
| 751 | 3802.29 | 37.64 |
| 752 | 3927.79 | 19.79 |
| 753 | 4008.95 | 23.57 |
| 754 | 4069.65 | 25.2 |
| 755 | 4115.03 | 21.09 |
| 756 | 4190.78 | 20.52 |
| 757 | 4191.13 | 33.64 |
| 758 | 4321.93 | 28.04 |
| 759 | 4334.88 | 20.38 |
| 760 | 4864.81 | 23.88 |
| 761 | 5043.12 | 26.54 |
| 762 | 5328.37 | 22.66 |
| 763 | 9704.8 | 20.79 |
| 764 | 9883.8 | 20.79 |
| 765 | 11738.36 | 19.6 |
| 766 | 11868.11 | 19.58 |
| 767 | 11883.99 | 19.6 |

With the present invention, it is possible to diagnose the rejection at a very early stage. Thus, the beginning rejection can be treated by medicaments at an early stage. The invention further enables an inexpensive, quick and reliable recognition of rejection with in part non-invasive or only minimal-invasive operations.

The migration time is determined by capillary electrophoresis (CE), for example, as set forth in the Example under item 2. In this Example, a glass capillary of 90 cm in length and with an inner diameter (ID) of 50 μm and an outer diameter (OD) of 360 μm is operated at an applied voltage of 25 or 30 kV. As the mobile solvent, 30% methanol, 0.5% formic acid or 20% acetonitrile and 0.25 M formic acid in water is used.

It is known that the CE migration times may vary. Nevertheless, the order in which the polypeptide markers are eluted is typically the same under the stated conditions for any CE system employed. In order to balance any differences in the migration time that may nevertheless occur, the system can be normalized using standards for which the migration times are exactly known. These standards may be, for example, the polypeptides stated in the Examples (see the Example, item 3).

The characterization of the polypeptides shown in Tables 1 to 2 was determined by means of capillary electrophoresis-mass spectrometry (CE-MS), a method which has been described in detail, for example, by Neuhoff et al. (Rapid communications in mass spectrometry, 2004, Vol. 20, pages 149-156). The variation of the molecular masses between individual measurements or between different mass spectrometers is relatively small when the calibration is exact, typically within a range of ±0.1%, preferably within a range of ±0.05%, more preferably ±0.03%.

The polypeptide markers according to the invention are proteins or peptides or degradation products of proteins or peptides. They may be chemically modified, for example, by posttranslational modifications, such as glycosylation, phosphorylation, alkylation or disulfide bridges, or by other reactions, for example, within the scope of degradation. In addition, the polypeptide markers may also be chemically altered, for example, oxidized, during the purification of the samples.

Proceeding from the parameters that determine the polypeptide markers (molecular weight and migration time), it is possible to identify the sequence of the corresponding polypeptides by methods known in the prior art.

The polypeptides according to the invention (see Tables 1 to 4) are used to diagnose a beginning rejection of the transplant. “Diagnosis” means the process of knowledge gaining by assigning symptoms or phenomena to a disease or injury. In the present case, rejection is concluded from the presence or absence of or differences in the amplitudes of particular polypeptide markers. Thus, the polypeptide markers according to the invention are determined in a sample from a subject, wherein their presence or absence and signal intensity/amplitude allow to conclude the existence of rejection. The presence or absence and amplitude of a polypeptide marker can be measured by any method known in the prior art. Methods which may be used are exemplified below.

A polypeptide marker is considered present if its measured value is at least as high as its threshold value. If the measured value is lower, then the polypeptide marker is considered absent. The threshold value can be determined either by the sensitivity of the measuring method (detection limit) or defined from experience.

In the context of the present invention, the threshold value is considered to be exceeded preferably if the measured value of the sample for a certain molecular mass is at least twice as high as that of a blank sample (for example, only buffer or solvent).

The polypeptide marker or markers is/are used in such a way that its/their presence or absence is measured, wherein the presence or absence is indicative of rejection (frequency markers No. 1 to 242; Table 2). Thus, there are polypeptide markers which are typically abundant in patients after kidney transplantation (NTx) and rejection, such as polypeptide markers No. 1 to 5, but less abundant in subjects after NTx without rejection (control). In addition, there are polypeptide markers which are abundant in subjects with no rejection, but less abundant or absent in subjects with rejection, for example, Nos. 6 to 15 (Table 2).

TABLE 2
Polypeptide markers (frequency markers) for the recognition of
rejection of kidney transplants, their molecular masses and migration
times as well as their presence and absence in groups of patients
after NTx with rejection and control groups, i.e. patients after
NTx without rejection, as a factor (1 = 100%, 0 = 0%;
sample processing and measurement as described in the Example).

| | Presence in | Presence in |
|---|---|---|
| | NTx with | NTx without |
| No. | rejection | rejection |
| 1 | 0.59 | 0.12 |
| 2 | 0.71 | 0.28 |
| 3 | 0.59 | 0.16 |
| 4 | 0.65 | 0.24 |
| 5 | 0.47 | 0.08 |
| 6 | 0.12 | 0.52 |
| 7 | 0.47 | 0.88 |
| 8 | 0.41 | 0.84 |
| 9 | 0.41 | 0.84 |
| 10 | 0.41 | 0.84 |
| 11 | 0.29 | 0.72 |
| 12 | 0.24 | 0.68 |
| 13 | 0.12 | 0.56 |
| 14 | 0.24 | 0.68 |
| 15 | 0.29 | 0.84 |
| 16 | 0.08 | 0.45 |
| 17 | 0.00 | 0.30 |
| 18 | 0.15 | 0.50 |
| 19 | 0.46 | 0.15 |
| 20 | 0.38 | 0.85 |
| 21 | 0.31 | 0.80 |
| 22 | 0.00 | 0.30 |
| 23 | 0.38 | 0.05 |
| 24 | 0.38 | 0.70 |
| 25 | 0.00 | 0.35 |
| 26 | 0.00 | 0.30 |
| 27 | 0.00 | 0.45 |
| 28 | 0.23 | 0.75 |
| 29 | 0.00 | 0.35 |
| 30 | 0.54 | 0.90 |
| 31 | 0.69 | 1.00 |
| 32 | 0.46 | 0.10 |
| 33 | 0.62 | 0.95 |
| 34 | 0.46 | 0.85 |
| 35 | 0.15 | 0.55 |
| 36 | 0.15 | 0.70 |
| 37 | 0.00 | 0.30 |
| 38 | 0.00 | 0.30 |
| 39 | 0.77 | 0.45 |
| 40 | 0.08 | 0.40 |
| 41 | 0.38 | 0.05 |
| 42 | 0.69 | 0.35 |
| 43 | 0.00 | 0.35 |
| 44 | 1.00 | 0.50 |
| 45 | 0.23 | 0.70 |
| 46 | 0.46 | 0.05 |
| 47 | 0.31 | 0.70 |
| 48 | 0.08 | 0.40 |
| 49 | 0.23 | 0.60 |
| 50 | 0.08 | 0.45 |
| 51 | 0.00 | 0.35 |
| 52 | 0.08 | 0.40 |
| 53 | 0.23 | 0.55 |
| 54 | 0.00 | 0.35 |
| 55 | 0.23 | 0.55 |
| 56 | 0.92 | 0.60 |
| 57 | 0.69 | 0.35 |
| 58 | 0.23 | 0.65 |
| 59 | 0.15 | 0.50 |
| 60 | 0.15 | 0.60 |
| 61 | 0.31 | 0.65 |
| 62 | 0.23 | 0.70 |
| 63 | 0.62 | 0.20 |
| 64 | 0.08 | 0.40 |
| 65 | 0.46 | 0.15 |
| 66 | 0.00 | 0.40 |
| 67 | 0.15 | 0.65 |
| 68 | 0.46 | 0.15 |
| 69 | 0.00 | 0.30 |
| 70 | 0.08 | 0.60 |
| 71 | 0.00 | 0.35 |
| 72 | 0.00 | 0.35 |
| 73 | 0.08 | 0.45 |
| 74 | 0.00 | 0.30 |
| 75 | 0.08 | 0.45 |
| 76 | 0.08 | 0.40 |
| 77 | 0.08 | 0.60 |
| 78 | 0.54 | 0.15 |
| 79 | 0.00 | 0.30 |
| 80 | 0.00 | 0.35 |
| 81 | 0.23 | 0.55 |
| 82 | 0.38 | 1.00 |
| 83 | 0.31 | 0.65 |
| 84 | 0.00 | 0.30 |
| 85 | 0.31 | 0.70 |
| 86 | 0.31 | 0.80 |
| 87 | 0.00 | 0.50 |
| 88 | 0.23 | 0.55 |
| 89 | 0.38 | 0.70 |
| 90 | 0.00 | 0.30 |
| 91 | 0.00 | 0.35 |
| 92 | 0.08 | 0.75 |
| 93 | 0.00 | 0.30 |
| 94 | 0.62 | 0.25 |
| 95 | 0.15 | 0.50 |
| 96 | 0.38 | 0.75 |
| 97 | 0.08 | 0.45 |
| 98 | 0.15 | 0.55 |
| 99 | 0.15 | 0.60 |
| 100 | 0.46 | 0.85 |
| 101 | 0.08 | 0.45 |
| 102 | 0.15 | 0.55 |
| 103 | 0.08 | 0.40 |
| 104 | 0.69 | 1.00 |
| 105 | 0.54 | 0.90 |
| 106 | 0.54 | 0.90 |
| 107 | 0.00 | 0.30 |
| 108 | 0.69 | 1.00 |
| 109 | 0.46 | 0.15 |
| 110 | 0.54 | 0.05 |
| 111 | 0.38 | 0.05 |
| 112 | 0.15 | 0.50 |
| 113 | 0.46 | 0.80 |
| 114 | 0.00 | 0.35 |
| 115 | 0.46 | 0.90 |
| 116 | 0.54 | 0.90 |
| 117 | 0.62 | 0.95 |
| 118 | 0.62 | 1.00 |
| 119 | 0.00 | 0.35 |
| 120 | 0.38 | 0.95 |
| 121 | 0.08 | 0.40 |
| 122 | 0.31 | 0.00 |
| 123 | 0.46 | 0.80 |
| 124 | 0.08 | 0.40 |
| 125 | 0.08 | 0.45 |
| 126 | 0.00 | 0.40 |
| 127 | 0.92 | 0.60 |
| 128 | 0.00 | 0.45 |
| 129 | 0.62 | 1.00 |
| 130 | 0.00 | 0.35 |
| 131 | 0.00 | 0.35 |
| 132 | 0.08 | 0.40 |
| 133 | 0.31 | 0.75 |
| 134 | 0.46 | 0.10 |
| 135 | 0.31 | 0.00 |
| 136 | 0.08 | 0.45 |
| 137 | 0.38 | 0.05 |
| 138 | 0.54 | 0.90 |
| 139 | 1.00 | 0.70 |
| 140 | 0.54 | 0.85 |
| 141 | 0.54 | 0.20 |
| 142 | 0.38 | 0.80 |
| 143 | 0.08 | 0.45 |
| 144 | 0.54 | 0.95 |
| 145 | 0.00 | 0.40 |
| 146 | 0.38 | 0.70 |
| 147 | 0.15 | 0.75 |
| 148 | 0.00 | 0.30 |
| 149 | 0.77 | 0.45 |
| 150 | 0.00 | 0.30 |
| 151 | 0.31 | 0.00 |
| 152 | 0.38 | 0.70 |
| 153 | 0.08 | 0.55 |
| 154 | 0.15 | 0.50 |
| 155 | 0.00 | 0.40 |
| 156 | 0.08 | 0.45 |
| 157 | 0.23 | 0.60 |
| 158 | 0.00 | 0.30 |
| 159 | 0.54 | 0.05 |
| 160 | 0.38 | 0.70 |
| 161 | 0.46 | 0.00 |
| 162 | 0.46 | 0.15 |
| 163 | 0.54 | 0.90 |
| 164 | 0.62 | 0.15 |
| 165 | 0.15 | 0.50 |
| 166 | 0.23 | 0.55 |
| 167 | 0.31 | 0.00 |
| 168 | 0.62 | 0.25 |
| 169 | 0.46 | 0.80 |
| 170 | 0.15 | 0.75 |
| 171 | 0.31 | 0.90 |
| 172 | 0.38 | 0.80 |
| 173 | 0.54 | 0.20 |
| 174 | 0.38 | 0.80 |
| 175 | 0.00 | 0.50 |
| 176 | 0.46 | 0.15 |
| 177 | 0.54 | 0.20 |
| 178 | 0.31 | 0.70 |
| 179 | 0.00 | 0.30 |
| 180 | 0.31 | 0.75 |
| 181 | 0.00 | 0.50 |
| 182 | 0.31 | 0.00 |
| 183 | 0.54 | 0.90 |
| 184 | 0.00 | 0.30 |
| 185 | 0.08 | 0.40 |
| 186 | 0.08 | 0.40 |
| 187 | 0.00 | 0.30 |
| 188 | 0.46 | 0.90 |
| 189 | 0.00 | 0.30 |
| 190 | 0.23 | 0.90 |
| 191 | 0.00 | 0.35 |
| 192 | 0.62 | 0.30 |
| 193 | 0.00 | 0.40 |
| 194 | 0.00 | 0.50 |
| 195 | 0.54 | 0.90 |
| 196 | 0.46 | 0.10 |
| 197 | 0.15 | 0.45 |
| 198 | 0.62 | 0.30 |
| 199 | 0.38 | 0.70 |
| 200 | 0.62 | 0.25 |
| 201 | 0.23 | 0.55 |
| 202 | 0.23 | 0.75 |
| 203 | 0.31 | 0.75 |
| 204 | 0.54 | 0.90 |
| 205 | 0.08 | 0.45 |
| 206 | 0.38 | 0.05 |
| 207 | 0.31 | 0.65 |
| 208 | 0.62 | 0.25 |
| 209 | 0.08 | 0.40 |
| 210 | 0.08 | 0.45 |
| 211 | 0.38 | 0.00 |
| 212 | 0.23 | 0.60 |
| 213 | 0.31 | 0.00 |
| 214 | 0.00 | 0.30 |
| 215 | 0.54 | 0.20 |
| 216 | 0.31 | 0.65 |
| 217 | 0.54 | 0.85 |
| 218 | 0.85 | 0.40 |
| 219 | 0.54 | 0.85 |
| 220 | 0.77 | 0.45 |
| 221 | 0.31 | 0.75 |
| 222 | 0.23 | 0.60 |
| 223 | 0.38 | 0.00 |
| 224 | 0.54 | 0.85 |
| 225 | 0.00 | 0.30 |
| 226 | 0.38 | 0.75 |
| 227 | 0.15 | 0.70 |
| 228 | 0.08 | 0.50 |
| 229 | 0.00 | 0.35 |
| 230 | 0.23 | 0.55 |
| 231 | 0.31 | 0.00 |
| 232 | 0.38 | 0.70 |
| 233 | 0.38 | 0.70 |
| 234 | 0.46 | 0.15 |
| 235 | 0.00 | 0.30 |
| 236 | 0.08 | 0.55 |
| 237 | 0.23 | 0.55 |
| 238 | 0.08 | 0.40 |
| 239 | 0.46 | 0.10 |
| 240 | 0.77 | 0.30 |
| 241 | 0.15 | 0.50 |
| 242 | 0.54 | 0.20 |

In addition or also alternatively to the frequency markers (determination of presence or absence), the amplitude markers as stated in Tables 3 and 4 may also be used for the diagnosis of rejections of kidney transplants (Nos. 243-767). Amplitude markers are used in such a way that the presence or absence is not critical, but the height of the signal (the amplitude) decides if the signal is present in both groups. Two normalization methods are possible to achieve comparability between differently concentrated samples or different measuring methods:

In the first approach, all peptide signals of a sample are normalized to a total amplitude of 1 million counts. Therefore, the respective mean amplitudes of the individual markers are stated as parts per million (ppm). The amplitude markers obtained by this method are shown in Table 3 (Nos. 243-627). In addition, it is possible to define further amplitude markers by an alternative normalization method:

In this case, all peptide signals of a sample are scaled with a common normalization factor. Thus, a linear regression between the peptide amplitudes of the individual samples and the reference values of known polypeptides in the sample is formed. The slope of the regression line just corresponds to the relative concentration and is used as a normalization factor for this sample. The markers which are characterized by this method are shown in Table 4.

All groups employed consist of at least 19, preferably at least 20, individual patient or control samples in order to obtain reliable mean amplitudes. The decision for a diagnosis (rejection of the kidney transplant or not) is made as a function of how high the amplitude of the respective polypeptide markers in the patient sample is in comparison with the mean amplitudes in the control groups or the rejection group. If the amplitude rather corresponds to the mean amplitudes of the rejection group, rejection of the kidney transplant is to be considered, and if it rather corresponds to the mean amplitudes of the control group, rejection is not to be considered. A more exact definition shall be given by means of marker No. 247 (Table 3). The mean amplitude of the marker is significantly increased when the kidney transplant is being rejected (654 ppm vs. 75 ppm in the group without rejection). Now, if the value for this marker in a patient sample is from 0 to 75 ppm or exceeds this range by a maximum of 20%, i.e., from 0 to 90 ppm, then this sample belongs to the control group without rejection. If the value is 654 ppm or up to 20% below, or higher, i.e., between 523 and very high values, rejection after kidney transplantation is to be considered.

TABLE 3
Amplitude markers (ppm normalization)

| | Mean | Mean |
|---|---|---|
| | amplitude | amplitude |
| | NTx with | NTx w/o |
| No. | rejection | rejection |
| 243 | 26 | 55 |
| 244 | 7 | 27 |
| 245 | 92 | 200 |
| 246 | 159 | 343 |
| 247 | 654 | 75 |
| 248 | 130 | 406 |
| 249 | 105 | 50 |
| 250 | 306 | 651 |
| 251 | 2018 | 692 |
| 252 | 71 | 31 |
| 253 | 140 | 58 |
| 254 | 187 | 32 |
| 255 | 36 | 102 |
| 256 | 1415 | 699 |
| 257 | 232 | 79 |
| 258 | 192 | 90 |
| 259 | 78 | 167 |
| 260 | 51 | 17 |
| 261 | 14 | 44 |
| 262 | 125 | 56 |
| 263 | 17 | 171 |
| 264 | 16 | 94 |
| 265 | 110 | 221 |
| 266 | 75 | 17 |
| 267 | 264 | 76 |
| 268 | 38 | 123 |
| 269 | 13 | 27 |
| 270 | 114 | 317 |
| 271 | 954 | 403 |
| 272 | 18 | 44 |
| 273 | 79 | 252 |
| 274 | 278 | 88 |
| 275 | 538 | 228 |
| 276 | 269 | 1311 |
| 277 | 242 | 47 |
| 278 | 40 | 84 |
| 279 | 3183 | 6781 |
| 280 | 427 | 1055 |
| 281 | 32 | 66 |
| 282 | 2586 | 6023 |
| 283 | 583 | 115 |
| 284 | 195 | 397 |
| 285 | 40 | 81 |
| 286 | 568 | 1293 |
| 287 | 511 | 2560 |
| 288 | 108 | 45 |
| 289 | 89 | 38 |
| 290 | 27 | 89 |
| 291 | 44 | 101 |
| 292 | 551 | 275 |
| 293 | 283 | 121 |
| 294 | 2555 | 5275 |
| 295 | 19 | 55 |
| 296 | 62 | 143 |
| 297 | 51 | 163 |
| 298 | 78 | 365 |
| 299 | 9 | 78 |
| 300 | 91 | 348 |
| 301 | 244 | 118 |
| 302 | 302 | 807 |
| 303 | 130 | 42 |
| 304 | 66 | 219 |
| 305 | 4715 | 10644 |
| 306 | 54 | 242 |
| 307 | 50 | 120 |
| 308 | 117 | 318 |
| 309 | 270 | 617 |
| 310 | 237 | 40 |
| 311 | 68 | 151 |
| 312 | 356 | 933 |
| 313 | 68 | 11 |
| 314 | 20 | 48 |
| 315 | 657 | 1399 |
| 316 | 86 | 38 |
| 317 | 90 | 193 |
| 318 | 46 | 95 |
| 319 | 2935 | 1299 |
| 320 | 117 | 36 |
| 321 | 2781 | 1300 |
| 322 | 1234 | 2727 |
| 323 | 82 | 228 |
| 324 | 183 | 47 |
| 325 | 25 | 58 |
| 326 | 98 | 252 |
| 327 | 221 | 540 |
| 328 | 189 | 412 |
| 329 | 127 | 39 |
| 330 | 125 | 416 |
| 331 | 435 | 880 |
| 332 | 72 | 235 |
| 333 | 31 | 74 |
| 334 | 126 | 62 |
| 335 | 246 | 77 |
| 336 | 692 | 310 |
| 337 | 203 | 90 |
| 338 | 98 | 43 |
| 339 | 268 | 850 |
| 340 | 457 | 106 |
| 341 | 193 | 397 |
| 342 | 225 | 79 |
| 343 | 173 | 349 |
| 344 | 59 | 122 |
| 345 | 167 | 79 |
| 346 | 83 | 181 |
| 347 | 569 | 152 |
| 348 | 165 | 462 |
| 349 | 61 | 139 |
| 350 | 345 | 168 |
| 351 | 77 | 237 |
| 352 | 96 | 47 |
| 353 | 59 | 138 |
| 354 | 29 | 98 |
| 355 | 19 | 41 |
| 356 | 134 | 60 |
| 357 | 248 | 122 |
| 358 | 453 | 211 |
| 359 | 272 | 70 |
| 360 | 592 | 271 |
| 361 | 1354 | 3025 |
| 362 | 138 | 63 |
| 363 | 71 | 334 |
| 364 | 207 | 48 |
| 365 | 57 | 18 |
| 366 | 32 | 112 |
| 367 | 14 | 94 |
| 368 | 71 | 196 |
| 369 | 158 | 646 |
| 370 | 129 | 45 |
| 371 | 251 | 523 |
| 372 | 180 | 388 |
| 373 | 21 | 89 |
| 374 | 934 | 450 |
| 375 | 225 | 592 |
| 376 | 178 | 705 |
| 377 | 10523 | 3845 |
| 378 | 152 | 459 |
| 379 | 98 | 383 |
| 380 | 506 | 137 |
| 381 | 38 | 152 |
| 382 | 45 | 180 |
| 383 | 68 | 285 |
| 384 | 54 | 211 |
| 385 | 487 | 1538 |
| 386 | 2496 | 891 |
| 387 | 42 | 92 |
| 388 | 318 | 82 |
| 389 | 82 | 40 |
| 390 | 170 | 407 |
| 391 | 1145 | 2429 |
| 392 | 11134 | 3717 |
| 393 | 157 | 398 |
| 394 | 21 | 53 |
| 395 | 149 | 38 |
| 396 | 89 | 220 |
| 397 | 866 | 336 |
| 398 | 1073 | 2666 |
| 399 | 300 | 1070 |
| 400 | 54 | 143 |
| 401 | 243 | 721 |
| 402 | 61 | 171 |
| 403 | 122 | 301 |
| 404 | 184 | 386 |
| 405 | 59 | 144 |
| 406 | 46 | 100 |
| 407 | 17 | 229 |
| 408 | 45 | 175 |
| 409 | 809 | 1621 |
| 410 | 62 | 142 |
| 411 | 303 | 793 |
| 412 | 273 | 83 |
| 413 | 84 | 20 |
| 414 | 105 | 228 |
| 415 | 75 | 26 |
| 416 | 35 | 97 |
| 417 | 173 | 63 |
| 418 | 329 | 708 |
| 419 | 65 | 147 |
| 420 | 42 | 377 |
| 421 | 28 | 222 |
| 422 | 1098 | 393 |
| 423 | 259 | 18 |
| 424 | 276 | 591 |
| 425 | 239 | 104 |
| 426 | 86 | 220 |
| 427 | 120 | 254 |
| 428 | 14 | 27 |
| 429 | 313 | 751 |
| 430 | 129 | 588 |
| 431 | 30 | 83 |
| 432 | 271 | 125 |
| 433 | 262 | 713 |
| 434 | 7705 | 3374 |
| 435 | 245 | 1124 |
| 436 | 158 | 509 |
| 437 | 151 | 612 |
| 438 | 67 | 12 |
| 439 | 1875 | 4720 |
| 440 | 207 | 95 |
| 441 | 445 | 206 |
| 442 | 38 | 108 |
| 443 | 1858 | 5175 |
| 444 | 1156 | 431 |
| 445 | 32 | 299 |
| 446 | 630 | 263 |
| 447 | 308 | 125 |
| 448 | 304 | 41 |
| 449 | 397 | 1003 |
| 450 | 88 | 275 |
| 451 | 126 | 273 |
| 452 | 266 | 112 |
| 453 | 487 | 231 |
| 454 | 2402 | 1196 |
| 455 | 42 | 161 |
| 456 | 79 | 159 |
| 457 | 173 | 576 |
| 458 | 126 | 61 |
| 459 | 157 | 434 |
| 460 | 162 | 65 |
| 461 | 76 | 167 |
| 462 | 3335 | 808 |
| 463 | 206 | 33 |
| 464 | 140 | 69 |
| 465 | 3493 | 1402 |
| 466 | 114 | 35 |
| 467 | 36 | 122 |
| 468 | 1401 | 395 |
| 469 | 53 | 184 |
| 470 | 105 | 286 |
| 471 | 228 | 481 |
| 472 | 320 | 710 |
| 473 | 173 | 555 |
| 474 | 31 | 65 |
| 475 | 155 | 34 |
| 476 | 78 | 452 |
| 477 | 51 | 102 |
| 478 | 49 | 136 |
| 479 | 277 | 134 |
| 480 | 312 | 732 |
| 481 | 99 | 38 |
| 482 | 90 | 33 |
| 483 | 112 | 241 |
| 484 | 262 | 879 |
| 485 | 253 | 539 |
| 486 | 599 | 125 |
| 487 | 428 | 192 |
| 488 | 119 | 339 |
| 489 | 409 | 189 |
| 490 | 77 | 27 |
| 491 | 267 | 133 |
| 492 | 148 | 666 |
| 493 | 2042 | 4248 |
| 494 | 838 | 213 |
| 495 | 474 | 206 |
| 496 | 55 | 144 |
| 497 | 62 | 243 |
| 498 | 187 | 82 |
| 499 | 148 | 323 |
| 500 | 403 | 98 |
| 501 | 567 | 1272 |
| 502 | 84 | 225 |
| 503 | 99 | 208 |
| 504 | 56 | 135 |
| 505 | 87 | 35 |
| 506 | 255 | 109 |
| 507 | 199 | 412 |
| 508 | 81 | 184 |
| 509 | 229 | 93 |
| 510 | 539 | 1166 |
| 511 | 1716 | 747 |
| 512 | 127 | 345 |
| 513 | 272 | 1163 |
| 514 | 242 | 118 |
| 515 | 1283 | 393 |
| 516 | 56 | 114 |
| 517 | 273 | 88 |
| 518 | 200 | 413 |
| 519 | 201 | 83 |
| 520 | 2028 | 672 |
| 521 | 46 | 22 |
| 522 | 396 | 1071 |
| 523 | 638 | 269 |
| 524 | 530 | 99 |
| 525 | 82 | 284 |
| 526 | 32 | 130 |
| 527 | 2392 | 811 |
| 528 | 581 | 208 |
| 529 | 739 | 312 |
| 530 | 226 | 93 |
| 531 | 85 | 212 |
| 532 | 668 | 298 |
| 533 | 522 | 1268 |
| 534 | 20 | 62 |
| 535 | 152 | 73 |
| 536 | 118 | 382 |
| 537 | 65 | 213 |
| 538 | 99 | 208 |
| 539 | 76 | 225 |
| 540 | 127 | 257 |
| 541 | 1908 | 711 |
| 542 | 371 | 772 |
| 543 | 292 | 607 |
| 544 | 128 | 61 |
| 545 | 144 | 315 |
| 546 | 349 | 124 |
| 547 | 209 | 505 |
| 548 | 189 | 87 |
| 549 | 74 | 158 |
| 550 | 49 | 148 |
| 551 | 795 | 1820 |
| 552 | 3458 | 7197 |
| 553 | 329 | 733 |
| 554 | 606 | 1268 |
| 555 | 1570 | 440 |
| 556 | 318 | 932 |
| 557 | 1300 | 2977 |
| 558 | 467 | 1089 |
| 559 | 357 | 166 |
| 560 | 145 | 311 |
| 561 | 1128 | 2318 |
| 562 | 243 | 117 |
| 563 | 29 | 178 |
| 564 | 113 | 274 |
| 565 | 340 | 131 |
| 566 | 369 | 907 |
| 567 | 125 | 257 |
| 568 | 329 | 137 |
| 569 | 488 | 230 |
| 570 | 255 | 96 |
| 571 | 1143 | 525 |
| 572 | 608 | 168 |
| 573 | 3236 | 12016 |
| 574 | 1815 | 3775 |
| 575 | 479 | 136 |
| 576 | 482 | 215 |
| 577 | 785 | 1916 |
| 578 | 1209 | 3253 |
| 579 | 33 | 77 |
| 580 | 884 | 425 |
| 581 | 661 | 227 |
| 582 | 258 | 124 |
| 583 | 63 | 172 |
| 584 | 214 | 735 |
| 585 | 75 | 170 |
| 586 | 117 | 272 |
| 587 | 405 | 152 |
| 588 | 136 | 68 |
| 589 | 1223 | 4112 |
| 590 | 393 | 834 |
| 591 | 152 | 67 |
| 592 | 323 | 651 |
| 593 | 68 | 159 |
| 594 | 2842 | 6695 |
| 595 | 1116 | 3083 |
| 596 | 197 | 39 |
| 597 | 445 | 1087 |
| 598 | 153 | 345 |
| 599 | 552 | 268 |
| 600 | 68 | 144 |
| 601 | 1061 | 467 |
| 602 | 465 | 170 |
| 603 | 393 | 804 |
| 604 | 237 | 86 |
| 605 | 552 | 1617 |
| 606 | 384 | 161 |
| 607 | 296 | 1123 |
| 608 | 928 | 402 |
| 609 | 570 | 186 |
| 610 | 134 | 57 |
| 611 | 188 | 449 |
| 612 | 2616 | 1130 |
| 613 | 5111 | 1486 |
| 614 | 501 | 216 |
| 615 | 612 | 249 |
| 616 | 5082 | 1136 |
| 617 | 177 | 75 |
| 618 | 975 | 331 |
| 619 | 314 | 120 |
| 620 | 523 | 129 |
| 621 | 948 | 341 |
| 622 | 129 | 43 |
| 623 | 344 | 115 |
| 624 | 379 | 111 |
| 625 | 269 | 119 |
| 626 | 672 | 214 |
| 627 | 784 | 346 |

TABLE 4
Amplitude markers (with normalization function)

| | Mean | Mean |
|---|---|---|
| | amplitude | amplitude |
| | NTx with | NTx w/o |
| No. | rejection | rejection |
| 628 | 102 | 46 |
| 629 | 42 | 132 |
| 630 | 59 | 128 |
| 631 | 58 | 121 |
| 632 | 167 | 368 |
| 633 | 322 | 706 |
| 634 | 130 | 59 |
| 635 | 93 | 194 |
| 636 | 74 | 210 |
| 637 | 106 | 50 |
| 638 | 23 | 171 |
| 639 | 422 | 181 |
| 640 | 441 | 215 |
| 641 | 210 | 443 |
| 642 | 85 | 203 |
| 643 | 4616 | 9285 |
| 644 | 3824 | 1492 |
| 645 | 2112 | 4529 |
| 646 | 297 | 166 |
| 647 | 342 | 151 |
| 648 | 1218 | 607 |
| 649 | 1611 | 509 |
| 650 | 54 | 327 |
| 651 | 109 | 356 |
| 652 | 1095 | 2614 |
| 653 | 3047 | 910 |
| 654 | 503 | 1264 |
| 655 | 329 | 671 |
| 656 | 238 | 49 |
| 657 | 69 | 167 |
| 658 | 560 | 140 |
| 659 | 51 | 124 |
| 660 | 135 | 280 |
| 661 | 424 | 1067 |
| 662 | 81 | 735 |
| 663 | 90 | 393 |
| 664 | 3844 | 1156 |
| 665 | 89 | 184 |
| 666 | 373 | 128 |
| 667 | 120 | 367 |
| 668 | 4356 | 1419 |
| 669 | 204 | 857 |
| 670 | 85 | 179 |
| 671 | 532 | 172 |
| 672 | 8706 | 18229 |
| 673 | 3028 | 1430 |
| 674 | 80 | 227 |
| 675 | 1072 | 2201 |
| 676 | 273 | 1863 |
| 677 | 154 | 412 |
| 678 | 3259 | 6642 |
| 679 | 123 | 306 |
| 680 | 610 | 205 |
| 681 | 711 | 2351 |
| 682 | 119 | 246 |
| 683 | 43 | 237 |
| 684 | 55 | 280 |
| 685 | 87 | 182 |
| 686 | 177 | 149 |
| 687 | 976 | 359 |
| 688 | 125 | 467 |
| 689 | 792 | 3523 |
| 690 | 1057 | 2318 |
| 691 | 2630 | 5672 |
| 692 | 715 | 208 |
| 693 | 253 | 535 |
| 694 | 221 | 571 |
| 695 | 102 | 273 |
| 696 | 226 | 470 |
| 697 | 4811 | 400 |
| 698 | 545 | 261 |
| 699 | 162 | 473 |
| 700 | 428 | 21 |
| 701 | 192 | 68 |
| 702 | 403 | 159 |
| 703 | 52 | 505 |
| 704 | 133 | 276 |
| 705 | 82 | 621 |
| 706 | 163 | 384 |
| 707 | 1485 | 632 |
| 708 | 930 | 362 |
| 709 | 73 | 147 |
| 710 | 417 | 91 |
| 711 | 2476 | 1043 |
| 712 | 51 | 188 |
| 713 | 500 | 238 |
| 714 | 494 | 211 |
| 715 | 372 | 50 |
| 716 | 202 | 59 |
| 717 | 176 | 84 |
| 718 | 528 | 1060 |
| 719 | 102210 | 34136 |
| 720 | 1718 | 511 |
| 721 | 594 | 210 |
| 722 | 965 | 386 |
| 723 | 72 | 181 |
| 724 | 1139 | 218 |
| 725 | 566 | 209 |
| 726 | 55 | 134 |
| 727 | 1917 | 696 |
| 728 | 129 | 265 |
| 729 | 227 | 84 |
| 730 | 141 | 62 |
| 731 | 125 | 302 |
| 732 | 1273 | 474 |
| 733 | 320 | 320 |
| 734 | 93 | 116 |
| 735 | 149 | 352 |
| 736 | 11770 | 5333 |
| 737 | 593 | 267 |
| 738 | 117 | 265 |
| 739 | 122 | 261 |
| 740 | 10614 | 2545 |
| 741 | 659 | 183 |
| 742 | 189 | 866 |
| 743 | 788 | 195 |
| 744 | 1416 | 703 |
| 745 | 910 | 404 |
| 746 | 347 | 149 |
| 747 | 121 | 258 |
| 748 | 116 | 264 |
| 749 | 219 | 784 |
| 750 | 1562 | 723 |
| 751 | 377 | 137 |
| 752 | 336 | 1099 |
| 753 | 3676 | 1031 |
| 754 | 1401 | 695 |
| 755 | 1148 | 551 |
| 756 | 310 | 906 |
| 757 | 164 | 429 |
| 758 | 945 | 303 |
| 759 | 975 | 1242 |
| 760 | 1077 | 730 |
| 761 | 1059 | 513 |
| 762 | 7419 | 1054 |
| 763 | 405 | 179 |
| 764 | 984 | 461 |
| 765 | 49075 | 22587 |
| 766 | 2522 | 864 |
| 767 | 569 | 479 |

The subject from whom the sample in which the presence or absence or the amplitude of one or more polypeptide markers is determined is derived may be any subject that is capable of suffering from rejection after NTx, for example, an animal or human. Preferably, the subject is a mammal, and most preferably it is a human.

In a preferred embodiment of the invention, not just one polypeptide marker, but a combination of markers, is used to diagnosis rejection after NTx, wherein the existence of rejection after NTx is concluded from their presence or absence and differences in the amplitude. By comparing a plurality of polypeptide markers, a bias in the overall result from a few individual deviations from the typical presence probability in the sick or control individual can be reduced or avoided.

The sample in which the presence or absence or amplitude of the polypeptide marker or markers according to the invention is measured may be any sample which is obtained from the body of the subject. The sample is a sample which has a polypeptide composition suitable for providing information about the state of the subject (rejection after NTx or not). For example, it may be blood, urine, synovial fluid, a tissue fluid, a body secretion, sweat, cerebrospinal fluid, lymph, intestinal, gastric or pancreatic juice, bile, lacrimal fluid, a tissue sample, sperm, vaginal fluid or a feces sample. Preferably, it is a liquid sample.

In a preferred embodiment, the sample is a urine sample or blood sample, and said blood sample may be a (blood) serum or (blood) plasma sample.

Urine samples can be taken as known in the prior art. Preferably, a midstream urine sample is used in the context of the present invention. For example, the urine sample may be taken by means of a catheter or also by means of an urination apparatus as described in WO 01/74275.

Blood samples can be taken by methods known in the prior art, for example, from a vein, artery or capillary. Usually, a blood sample is obtained by withdrawing venous blood by means of a syringe, for example, from an arm of the subject. The term “blood sample” includes samples obtained from blood by further purification and separation methods, such as blood plasma or blood serum.

The presence or absence and amplitude of a polypeptide marker in the sample may be determined by any method known in the prior art that is suitable for measuring polypeptide markers. Such methods are known to the skilled person. In principle, the presence or absence and amplitude of a polypeptide marker can be determined by direct methods, such as mass spectrometry, or indirect methods, for example, by means of ligands.

If required or desirable, the sample from the subject, for example, the urine sample, may be pretreated by any suitable means and, for example, purified or separated before the presence or absence and amplitude of the polypeptide marker or markers is measured. The treatment may comprise, for example, purification, separation, dilution or concentration. The methods may be, for example, centrifugation, filtration, ultrafiltration, dialysis, precipitation or chromatographic methods, such as affinity separation or separation by means of ion-exchange chromatography, or electrophoretic separation. Particular examples thereof are gel electrophoresis, two-dimensional polyacrylamide gel electrophoresis (2D-PAGE), capillary electrophoresis, metal affinity chromatography, immobilized metal affinity chromatography (IMAC), lectin-based affinity chromatography, liquid chromatography, high-performance liquid chromatography (HPLC), normal and reverse-phase HPLC, cation-exchange chromatography and selective binding to surfaces. All these methods are well known to the skilled person, and the skilled person will be able to select the method as a function of the sample employed and the method for determining the presence or absence of the polypeptide marker or markers.

In one embodiment of the invention, the sample, before being measured, is separated by capillary electrophoresis, purified by ultracentrifugation and/or divided by ultrafiltration into fractions which contain polypeptide markers of a particular molecular size.

Preferably, a mass-spectrometric method is used to determine the presence or absence and amplitude of a polypeptide marker, wherein a purification or separation of the sample may be performed upstream from such method. As compared to the currently employed methods, mass-spectrometric analysis has the advantage that the concentration of many (>100) polypeptides of a sample can be determined by a single analysis. Any type of mass spectrometer may be employed. By means of mass spectrometry, it is possible to measure 10 fmol of a polypeptide marker, i.e., 0.1 ng of a 10 kDa protein, as a matter of routine with a measuring accuracy of about ±0.01% in a complex mixture. In mass spectrometers, an ion-forming unit is coupled with a suitable analytic device. For example, electrospray-ionization (ESI) interfaces are mostly used to measure ions in liquid samples, whereas the matrix-assisted laser desorption/ionization (MALDI) technique is used for measuring ions from a sample crystallized with a matrix. For analyzing the ions formed, quadrupoles, ion traps or time-of-flight (TOF) analyzers may be used.

In electrospray ionization (ESI), the molecules present in solution are atomized, inter alia, under the influence of high voltage (e.g., 1-8 kV), which forms charged droplets that become smaller from the evaporation of the solvent. Finally, so-called Coulomb explosions cause the formation of free ions, which can then be analyzed and detected.

In the analysis of the ions by means of TOF, a particular acceleration voltage is applied which confers an equal amount of kinetic energy to the ions. Thereafter, the time that the respective ions take to travel a particular drifting distance through the flying tube is measured very accurately. Since with equal amounts of kinetic energy, the velocity of the ions depends on their mass, the latter can thus be determined. TOF analyzers have a very high scanning speed and therefore reach a very high resolution.

Preferred methods for the determination of the presence or absence and amplitude of polypeptide markers include gas-phase ion spectrometry, such as laser desorption/ionization mass spectrometry, MALDI-TOF MS, SELDI-TOF MS (surfaceenhanced laser desorption/ionization), LC-MS (liquid chromatography/mass spectrometry), 2D-PAGE/MS and capillary electrophoresis-mass spectrometry (CE-MS). All methods mentioned are known to the skilled person.

A particularly preferred method is CE-MS, in which capillary electrophoresis is coupled with mass spectrometry. This method has been described in some detail, for example, in the German Patent Application DE 10021737, in Kaiser et al. (J Chromatogr A, 2003, Vol. 1013: 157-171, and Electrophoresis, 2004, 25: 2044-2055) and in Wittke et al. (Journal of Chromatography A, 2003, 1013: 173-181). The CE-MS technology allows one to determine the presence and amplitude of some hundreds of polypeptide markers of a sample simultaneously within a short time and in a small volume with high sensitivity. After a sample has been measured, a pattern of the measured polypeptide markers is prepared. This pattern can be compared with reference patterns of sick or healthy subjects. In most cases, it is sufficient to use a limited number of polypeptide markers for recognizing rejection after NTx. A CE-MS method which includes CE coupled on-line to an ESI-TOF MS device is further preferred.

For CE-MS, the use of volatile solvents is preferred, and it is best to work under essentially salt-free conditions. Examples of suitable solvents include acetonitrile, methanol and the like. The solvents can be diluted with water or admixed with a weak acid (e.g., from 0.1% to 1% formic acid) in order to protonate the analyte, preferably the polypeptides.

By means of capillary electrophoresis, it is possible to separate molecules by their charge and size. Neutral particles will migrate at the speed of the electroosmotic flow upon application of a current, while cations are accelerated towards the cathode, and anions are delayed. The advantage of capillaries in electrophoresis resides in their favorable ratio of surface to volume, which enables a good dissipation of the Joule heat generated during the current flow. This in turn allows high voltages (usually up to 30 kV) to be applied and thus a high separating performance and short times of analysis.

In capillary electrophoresis, silica glass capillaries having inner diameters of typically from 50 to 75 μm are usually employed. The lengths employed are from 30 to 100 cm. In addition, the capillaries are usually made of plastic-coated silica glass. The capillaries may be both untreated, i.e., expose their hydrophilic groups on the interior surface, or coated on the interior surface. A hydrophobic coating may be used to improve the resolution. In addition to the voltage, a pressure may also be applied, which typically is within a range of from 0 to 1 psi. The pressure may also be applied only during the performance or altered meanwhile.

In a preferred method for measuring polypeptide markers, the markers of the sample are separated by means of capillary electrophoresis, then directly ionized and transferred on-line to a mass spectrometer coupled thereto for detection.

In the method according to the invention, it is advantageous to use several polypeptide markers for recognizing rejection after NTx. In particular, at least three polypeptide markers may be used, for example, markers 1, 2 and 3; 1, 2 and 4; etc.

More preferred is the use of at least 4, 5 or 6 markers.

Even more preferred is the use of at least 10 markers, for example, markers 1 to 10.

Most preferred is the use of all 767 markers listed in Tables 1 and 2.

In order to determine the probability of the existence of rejection after NTx when several markers are used, statistic methods known to the skilled person may be used. For example, the Random Forests method described by Weissinger et al. (Kidney Int., 2004, 65: 2426-2434) may be used by using a computer program such as S-Plus.

### EXAMPLE

### 1. Sample Preparation

For detecting the polypeptide markers for recognizing rejection after NTx, urine was employed. Urine was withdrawn from patients after NTx with rejection as well as from patients after NTx without rejection (control group).

The clinical data for the patients of both groups at the time of transplantation are shown in the following Table 5:

TABLE 5
Clinical data for the groups of patients employed during transplantation

| | Control group | Group |
|---|---|---|
| | NTx without rejection | NTx with rejection |
| Number of patients | 29 | 19 |
| Age [years] | 55.6 ± 14.2 | 48.6 ± 14.0 |
| Sex [number m/f] | 19/10 | 10/9 |
| Number of patients with | 1 | 0 |
| former Tx | | |
| Origin of transplant: | | |
| Donor deceased | 28 | 17 |
| Living donor | 1 | 2 |
| Age of donors | 57.6 ± 14.5 | 47.3 ± 20.7 |
| Sex of donors [m/f] | 15/14 | 8/11 |

For the subsequent CE-MS measurement, the proteins which are also contained in the urine of patients in a higher concentration, such as albumin and immunoglobulins, had to be separated off by ultrafiltration. Thus, 700 μl of urine was removed and admixed with 700 ml of filtration buffer (2 M urea, 10 mM ammonia, 0.02% SDS). This 1.4 ml of sample volume was ultrafiltrated (20 kDa, Sartorius, GOttingen, Germany). The ultrafiltration was performed at 3000 rpm in a centrifuge until 1.1 ml of ultrafiltrate was obtained.

The 1.1 ml of filtrate obtained was then applied to a PD-10 column (Amersham Bioscience, Uppsala, Sweden) for desalting and eluted with 2.5 ml of 0.01% NH₄OH in water, and the eluate was subsequently lyophilized. For the CE-MS measurement, the polypeptides were then resuspended with 20 μl of water (HPLC grade, Merck).

### 2. CE-MS Measurement

The CE-MS measurements were performed with a capillary electrophoresis system from Beckman Coulter (P/ACE MDQ System; Beckman Coulter Inc., Fullerton, USA) and an ESI-TOF mass spectrometer from Bruker (micro-TOF MS, Bruker Daltonik, Bremen, Germany).

The CE capillaries were supplied by Beckman Coulter and had an ID/OD of 50/360 μm and a length of 90 cm. The mobile phase for the CE separation consisted of 20% acetonitrile and 0.25 M formic acid in water. For the “sheath flow” on the MS, 30% isopropanol with 0.5% formic acid was used at a flow rate of 2 μl/min. The coupling of CE and MS was realized by a CE-ESI-MS Sprayer Kit (Agilent Technologies, Waldbronn, Germany).

For injecting the sample, a pressure of from 1 to a maximum of 6 psi was applied, and the duration of the injection was 99 seconds. With these parameters, about 150 nl of the sample was injected into the capillary, which corresponds to about 10% of the capillary volume. A stacking technique was used to concentrate the sample in the capillary. Thus, before the sample was injected, a 1 M NH₃ solution was injected for 7 seconds (at 1 psi), and after the sample was injected, a 2 M formic acid solution was injected for 5 seconds. After the separation voltage (30 kV) was applied, the analytes were automatically concentrated between these solutions.

The subsequent CE separation was performed with a pressure method: 40 minutes at 0 psi, then 0.1 psi for 2 min, 0.2 psi for 2 min, 0.3 psi for 2 min, 0.4 psi for 2 min, and finally 0.5 psi for 32 min. The total duration of a separation run was thus 80 minutes.

In order to obtain as good as possible signal intensity on the side of the MS, the nebulizer gas was set to the lowest possible value. The voltage applied to the spray needle for generating the electrospray was 3700-4100 V. The remaining settings at the mass spectrometer were optimized for peptide detection according to the manufacturer's protocol. The spectra were recorded over a mass range of m/z 400 to m/z 3000 and accumulated every 3 seconds.

### 3. Standards for the CE Measurement

For checking and calibrating the CE measurement, the following proteins or polypeptides which are characterized by the stated CE migration times under the selected conditions were employed:

| Protein/polypeptide | CE Migration time |
|---|---|
| Aprotinin (SIGMA, Taufkirchen, DE, Cat. # A1153) | 9.2 min |
| Ribonuclease (SIGMA, Taufkirchen, DE, Cat. # R4875) | 10.9 min |
| Lysozyme (SIGMA, Taufkirchen, DE, Cat. # L7651) | 8.9 min |
| ″REV″, Sequence: REVQSKIGYGRQIIS | 15.6 min |
| ″ELM″, Sequence: ELMTGELPYSHINNRDQIIFMVGR | 23.4 min |
| ″KINCON″, Sequence: TGSLPYSHIGSRDQIIFMVGR | 20.0 min |
| ″GIVLY″ Sequence: GIVLYELMTGELPYSHIN | 36.8 min |

The proteins/polypeptides were each employed at a concentration of 10 pmol/μl in water. “REV”, “ELM, “KINCON” and “GIVLY” are synthetic peptides.

The molecular masses of the peptides and the m/z ratios of the individual charge states visible in MS are listed in the following Table:

| | H (mono) | | | | | | |
|---|---|---|---|---|---|---|---|
| | 1.0079 | 1.0079 | 1.0079 | 1.0079 | 1.0079 | 1.0079 | 1.0079 |
| | Aprotinin | Ribonuclease | Lysozyme | REV | KINCON | ELM | GIVLY |
| | Mono | Mono | Mono | Mono | Mono | Mono | Mono |
| m/z | Mass | Mass | Mass | Mass | Mass | Mass | Mass |
| 0 | 6513.09 | 13681.32 | 14303.88 | 1732.96 | 2333.19 | 2832.41 | 2048.03 |
| 1 | 6514.0979 | 13682.328 | 14304.888 | 1733.9679 | 2334.1979 | 2833.4179 | 2049.0379 |
| 2 | 3257.5529 | 6841.6679 | 7152.9479 | 867.4879 | 1167.6029 | 1417.2129 | 1025.0229 |
| 3 | 2172.0379 | 4561.4479 | 4768.9679 | 578.6612 | 778.7379 | 945.1446 | 683.6846 |
| 4 | 1629.2804 | 3421.3379 | 3576.9779 | 434.2479 | 584.3054 | 709.1104 | 513.0154 |
| 5 | 1303.6259 | 2737.2719 | 2861.7839 | 347.5999 | 467.6459 | 567.4899 | 410.6139 |
| 6 | 1086.5229 | 2281.2279 | 2384.9879 | 289.8346 | 389.8729 | 473.0762 | 342.3462 |
| 7 | 931.4494 | 1955.4822 | 2044.4193 | 248.5736 | 334.3208 | 405.6379 | 293.5836 |
| 8 | 815.1442 | 1711.1729 | 1788.9929 | 217.6279 | 292.6567 | 355.0592 | 257.0117 |
| 9 | 724.6846 | 1521.1546 | 1590.3279 | 193.559 | 260.2512 | 315.7201 | 228.5668 |
| 10 | 652.3169 | 1369.1399 | 1431.3959 | 174.3039 | 234.3269 | 284.2489 | 205.8109 |
| 11 | 593.107 | 1244.7643 | 1301.3606 | 158.5497 | 213.1161 | 258.4997 | 187.1924 |
| 12 | 543.7654 | 1141.1179 | 1192.9979 | 145.4212 | 195.4404 | 237.0421 | 171.6771 |
| 13 | 502.0148 | 1053.4171 | 1101.3063 | 134.3125 | 180.4841 | 218.8856 | 158.5486 |

What is claimed is:

1. A method for diagnosing the probability of rejection of a kidney after said kidney has been transplanted (NTx) in a subject patient, comprising:
obtaining a urine sample from a subject patient;
purifying said urine sample to remove high concentration proteins;
separating said urine sample into a plurality of polypeptides;
identifying said plurality of polypeptides based on the separation characteristics of each of said plurality of polypeptides in the separating step;
comparing said plurality of polypeptides to known polypeptide markers taken from control subjects with rejection NTx and without rejection after NTx to obtain a subset of polypeptide markers from said plurality of polypeptides that substantially match said known polypeptide markers;
wherein said known polypeptide markers are characterized by the following molecular masses and migration times (CE time):

| | Mass | CE time |
|---|---|---|
| Number | (Da) | (min) |
| 1 | 2078.9 | 36.2 |
| 2 | 1168.6 | 40.8 |
| 3 | 2121.1 | 38 |
| 4 | 3359.6 | 48.1 |
| 5 | 1707.8 | 37.7 |
| 6 | 2815.9 | 36 |
| 7 | 3516.1 | 30 |
| 8 | 1697.9 | 34.7 |
| 9 | 2838.9 | 37.8 |
| 10 | 3482.1 | 31.4 |
| 11 | 8052.3 | 27.8 |
| 12 | 1027.6 | 35.8 |
| 13 | 1031.6 | 37.5 |
| 14 | 3240.2 | 34 |
| 15 | 1811 | 42.3 |
| 16 | 1016.46 | 33.49 |
| 17 | 1026.47 | 35.31 |
| 18 | 1032.45 | 33.66 |
| 19 | 1059.69 | 33.55 |
| 20 | 1065.55 | 25.50 |
| 21 | 1078.52 | 27.75 |
| 22 | 1100.46 | 24.80 |
| 23 | 1100.58 | 21.83 |
| 24 | 1110.43 | 33.78 |
| 25 | 1112.56 | 23.99 |
| 26 | 1125.55 | 24.64 |
| 27 | 1132.61 | 23.70 |
| 28 | 1137.58 | 26.41 |
| 29 | 1137.67 | 28.70 |
| 30 | 1141.57 | 24.65 |
| 31 | 1150.61 | 22.43 |
| 32 | 1159.64 | 26.05 |
| 33 | 1162.59 | 20.21 |
| 34 | 1171.55 | 29.24 |
| 35 | 1178.6 | 26.84 |
| 36 | 1180.56 | 35.61 |
| 37 | 1185.59 | 26.38 |
| 38 | 1195.57 | 37.51 |
| 39 | 1197.56 | 21.09 |
| 40 | 1202.53 | 35.63 |
| 41 | 1211.69 | 21.38 |
| 42 | 1212.68 | 21.43 |
| 43 | 1217.6 | 39.21 |
| 44 | 1226.58 | 21.02 |
| 45 | 1232.59 | 24.37 |
| 46 | 1238.55 | 36.01 |
| 47 | 1239.54 | 35.39 |
| 48 | 1240.61 | 23.68 |
| 49 | 1245.81 | 33.86 |
| 50 | 1255.53 | 35.72 |
| 51 | 1263.61 | 19.74 |
| 52 | 1273.65 | 27.06 |
| 53 | 1276.75 | 19.89 |
| 54 | 1279.69 | 19.68 |
| 55 | 1285.63 | 28.14 |
| 56 | 1287.65 | 21.85 |
| 57 | 1297.77 | 21.90 |
| 58 | 1324.63 | 24.80 |
| 59 | 1348.68 | 20.23 |
| 60 | 1351.69 | 38.75 |
| 61 | 1357.67 | 22.44 |
| 62 | 1358.66 | 26.84 |
| 63 | 1379.68 | 28.05 |
| 64 | 1384.71 | 38.77 |
| 65 | 1400.72 | 22.44 |
| 66 | 1404.91 | 34.89 |
| 67 | 1405.71 | 20.16 |
| 68 | 1422.81 | 34.49 |
| 69 | 1426.71 | 19.92 |
| 70 | 1434.76 | 22.56 |
| 71 | 1437.72 | 30.40 |
| 72 | 1441.67 | 19.94 |
| 73 | 1441.73 | 28.87 |
| 74 | 1443.72 | 20.46 |
| 75 | 1446.68 | 19.74 |
| 76 | 1451.69 | 36.04 |
| 77 | 1456.71 | 29.28 |
| 78 | 1457.68 | 21.95 |
| 79 | 1462.67 | 39.43 |
| 80 | 1462.75 | 22.81 |
| 81 | 1466.72 | 28.51 |
| 82 | 1494.72 | 30.40 |
| 83 | 1513.7 | 29.29 |
| 84 | 1539.92 | 33.44 |
| 85 | 1545.75 | 29.24 |
| 86 | 1554.72 | 28.64 |
| 87 | 1561.77 | 29.45 |
| 88 | 1583.76 | 30.11 |
| 89 | 1584.77 | 29.72 |
| 90 | 1587.8 | 19.91 |
| 91 | 1621.78 | 30.05 |
| 92 | 1633.73 | 31.93 |
| 93 | 1635.72 | 37.66 |
| 94 | 1652.83 | 22.64 |
| 95 | 1658.8 | 29.91 |
| 96 | 1666.84 | 30.65 |
| 97 | 1674.82 | 23.24 |
| 98 | 1678.83 | 30.84 |
| 99 | 1688.76 | 20.22 |
| 100 | 1689.86 | 27.59 |
| 101 | 1692.86 | 32.09 |
| 102 | 1711.72 | 22.68 |
| 103 | 1713.83 | 30.77 |
| 104 | 1732.84 | 28.21 |
| 105 | 1734.87 | 23.61 |
| 106 | 1741.81 | 30.21 |
| 107 | 1749.85 | 20.00 |
| 108 | 1749.88 | 30.54 |
| 109 | 1764.86 | 29.88 |
| 110 | 1768.9 | 20.77 |
| 111 | 1783.86 | 39.97 |
| 112 | 1784.88 | 39.94 |
| 113 | 1809.91 | 20.98 |
| 114 | 1812.9 | 39.98 |
| 115 | 1813.78 | 31.87 |
| 116 | 1814.84 | 30.49 |
| 117 | 1822.81 | 30.87 |
| 118 | 1825.87 | 31.80 |
| 119 | 1832.92 | 31.91 |
| 120 | 1837.88 | 30.54 |
| 121 | 1852.85 | 31.25 |
| 122 | 1882.9 | 22.09 |
| 123 | 1885.95 | 21.25 |
| 124 | 1889.97 | 19.38 |
| 125 | 1930.94 | 20.67 |
| 126 | 1930.97 | 31.53 |
| 127 | 1938.96 | 21.39 |
| 128 | 1951.93 | 32.08 |
| 129 | 2059.02 | 23.12 |
| 130 | 2066.98 | 25.11 |
| 131 | 2096.91 | 20.23 |
| 132 | 2119.07 | 25.45 |
| 133 | 2124.94 | 20.39 |
| 134 | 2155.06 | 27.54 |
| 135 | 2158.12 | 22.08 |
| 136 | 2178.1 | 34.07 |
| 137 | 2189.08 | 27.17 |
| 138 | 2192.07 | 22.37 |
| 139 | 2233.14 | 20.52 |
| 140 | 2274.11 | 33.47 |
| 141 | 2286.06 | 25.98 |
| 142 | 2289.47 | 33.56 |
| 143 | 2305.65 | 34.65 |
| 144 | 2308.1 | 27.32 |
| 145 | 2313.11 | 33.57 |
| 146 | 2320.16 | 20.73 |
| 147 | 2349.14 | 27.36 |
| 148 | 2353.83 | 27.12 |
| 149 | 2356.74 | 35.53 |
| 150 | 2420.11 | 34.09 |
| 151 | 2459.17 | 34.05 |
| 152 | 2471.25 | 34.69 |
| 153 | 2474.24 | 19.77 |
| 154 | 2483.28 | 29.20 |
| 155 | 2487.18 | 25.95 |
| 156 | 2501.84 | 20.05 |
| 157 | 2524.62 | 19.81 |
| 158 | 2544.23 | 26.11 |
| 159 | 2564.77 | 27.19 |
| 160 | 2600.93 | 34.67 |
| 161 | 2658.1 | 21.45 |
| 162 | 2680.08 | 27.98 |
| 163 | 2686.97 | 29.06 |
| 164 | 2695.41 | 27.37 |
| 165 | 2713.31 | 29.13 |
| 166 | 2726.37 | 42.97 |
| 167 | 2728.65 | 29.05 |
| 168 | 2825.34 | 26.86 |
| 169 | 2854.27 | 21.54 |
| 170 | 2875.09 | 29.21 |
| 171 | 2891.43 | 29.40 |
| 172 | 2940.95 | 29.07 |
| 173 | 3001.26 | 37.28 |
| 174 | 3002.23 | 23.80 |
| 175 | 3005.84 | 24.48 |
| 176 | 3021.47 | 35.35 |
| 177 | 3023.05 | 37.20 |
| 178 | 3031.39 | 35.93 |
| 179 | 3046.47 | 20.64 |
| 180 | 3047.99 | 29.45 |
| 181 | 3048.04 | 35.82 |
| 182 | 3054.49 | 22.03 |
| 183 | 3108.55 | 31.22 |
| 184 | 3124.19 | 19.82 |
| 185 | 3182 | 30.72 |
| 186 | 3185.29 | 30.71 |
| 187 | 3238.04 | 29.05 |
| 188 | 3264.75 | 30.51 |
| 189 | 3272.4 | 36.47 |
| 190 | 3276.21 | 33.32 |
| 191 | 3344.47 | 20.95 |
| 192 | 3356.59 | 22.08 |
| 193 | 3378.05 | 38.81 |
| 194 | 3378.33 | 31.44 |
| 195 | 3400.13 | 31.50 |
| 196 | 3479.24 | 33.46 |
| 197 | 3515.51 | 23.23 |
| 198 | 3515.81 | 20.63 |
| 199 | 3536.54 | 20.35 |
| 200 | 3552.27 | 20.67 |
| 201 | 3576.5 | 21.47 |
| 202 | 3612.65 | 23.03 |
| 203 | 3617.74 | 26.97 |
| 204 | 3633.69 | 26.99 |
| 205 | 3657.59 | 23.19 |
| 206 | 3735.52 | 27.10 |
| 207 | 3798.16 | 20.18 |
| 208 | 3802.27 | 37.66 |
| 209 | 3805.59 | 27.63 |
| 210 | 3870.85 | 33.39 |
| 211 | 3890.96 | 27.27 |
| 212 | 3927.78 | 19.76 |
| 213 | 3968.58 | 25.01 |
| 214 | 3988.76 | 27.65 |
| 215 | 3991.47 | 34.46 |
| 216 | 4008.93 | 23.43 |
| 217 | 4015.99 | 28.02 |
| 218 | 4022.38 | 32.97 |
| 219 | 4041.27 | 20.42 |
| 220 | 4062.7 | 33.07 |
| 221 | 4101.34 | 28.51 |
| 222 | 4122.34 | 20.11 |
| 223 | 4160.09 | 28.82 |
| 224 | 4190.81 | 20.55 |
| 225 | 4236.87 | 24.02 |
| 226 | 4387.08 | 21.01 |
| 227 | 4584.13 | 24.18 |
| 228 | 4601.03 | 23.62 |
| 229 | 4624.83 | 19.89 |
| 230 | 4777.58 | 21.17 |
| 231 | 5000.89 | 30.20 |
| 232 | 5042.57 | 20.20 |
| 233 | 5143.4 | 20.17 |
| 234 | 5175.89 | 21.70 |
| 235 | 5340.29 | 25.10 |
| 236 | 5411.46 | 20.13 |
| 237 | 5831.21 | 23.80 |
| 238 | 6074.38 | 23.25 |
| 239 | 6629.99 | 27.15 |
| 240 | 7907.23 | 19.59 |
| 241 | 9972.75 | 19.67 |
| 242 | 11777.18 | 19.55 |

wherein said CE times are based on capillary electrophoresis using a glass capillary of 90 cm in length with an inner diameter (ID) of 50 μm at an applied voltage of 25 kV,
wherein 20% acetonitrile, 0.25 M formic acid in water is used as the mobile solvent for the capillary electrophoresis,
wherein said CE times are calibrated relative to the following values:

| Protein/Polypeptide | Migration time (minutes) |
|---|---|
| Aprotinin | 9.2 |
| Ribonuclease | 10.9 |
| Lysozyme | 8.9 |
| “REV” SEQ ID NO: 1 | 15.6 |
| “ELM” SEQ ID NO: 2 | 23.4 |
| “KINCON” SEQ ID NO: 3 | 20.0 |
| “GIVLY” SEQ ID NO: 4 | 36.8 |

determining if said subset of polypeptide markers comprises at least:
a first biomarker selected from the group consisting of: (1) markers 1 to 242 (frequency markers) having (a) a frequency of presence in patients with rejection after NTx of at least 0.31 and (b) a frequency of presence in patients with rejection after NTx at least two (2) times the frequency of presence in patients without rejection after NTx; and (2) markers 1 to 242 (frequency markers) having (a) a frequency of presence in patients without rejection after NTx of at least 0.31 and (b) a frequency of presence in patients without rejection after NTx at least two (2) times the frequency of presence in patients with rejection after NTx,
a second biomarker which is different than said first biomarker and is at least one selected from the group consisting of: (1) markers 1 to 242 (frequency markers) having (a) a frequency of presence in patients with rejection after NTx of at least 0.31 and (b) a frequency of presence in patients with rejection after NTx at least two (2) times the frequency of presence in patients without rejection after NTx; and (2) markers 1 to 242 (frequency markers) having (a) a frequency of presence in patients without rejection after NTx of at least 0.31 and (b) a frequency of presence in patients without rejection after NTx at least two (2) times the frequency of presence in patients with rejection after NTx, and
a third biomarker which is different than said first and second biomarkers and is at least one selected from the group consisting of markers 1 to 242 (frequency markers);
comparing the frequency of presence of said at least said first, second and third biomarkers in said urine sample of said subject patient to the frequency of presence of the same first, second and third biomarkers from said known polypeptide markers taken from said control subjects,
ranking said subject patient between said control subjects with rejection after NTx and without rejection after NTx based on the second comparing step; and
diagnosing the probability of rejection after NTx in the subject patient based on said ranking.

2. The method according to claim 1, wherein said determining, said second comparing and said ranking are made with the following reference values:

| | Presence in | Presence in |
|---|---|---|
| | NTx with | NTx without |
| No. | rejection | rejection |
| 1 | 0.59 | 0.12 |
| 2 | 0.71 | 0.28 |
| 3 | 0.59 | 0.16 |
| 4 | 0.65 | 0.24 |
| 5 | 0.47 | 0.08 |
| 6 | 0.12 | 0.52 |
| 7 | 0.47 | 0.88 |
| 8 | 0.41 | 0.84 |
| 9 | 0.41 | 0.84 |
| 10 | 0.41 | 0.84 |
| 11 | 0.29 | 0.72 |
| 12 | 0.24 | 0.68 |
| 13 | 0.12 | 0.56 |
| 14 | 0.24 | 0.68 |
| 15 | 0.29 | 0.84 |
| 16 | 0.08 | 0.45 |
| 17 | 0.00 | 0.30 |
| 18 | 0.15 | 0.50 |
| 19 | 0.46 | 0.15 |
| 20 | 0.38 | 0.85 |
| 21 | 0.31 | 0.80 |
| 22 | 0.00 | 0.30 |
| 23 | 0.38 | 0.05 |
| 24 | 0.38 | 0.70 |
| 25 | 0.00 | 0.35 |
| 26 | 0.00 | 0.30 |
| 27 | 0.00 | 0.45 |
| 28 | 0.23 | 0.75 |
| 29 | 0.00 | 0.35 |
| 30 | 0.54 | 0.90 |
| 31 | 0.69 | 1.00 |
| 32 | 0.46 | 0.10 |
| 33 | 0.62 | 0.95 |
| 34 | 0.46 | 0.85 |
| 35 | 0.15 | 0.55 |
| 36 | 0.15 | 0.70 |
| 37 | 0.00 | 0.30 |
| 38 | 0.00 | 0.30 |
| 39 | 0.77 | 0.45 |
| 40 | 0.08 | 0.40 |
| 41 | 0.38 | 0.05 |
| 42 | 0.69 | 0.35 |
| 43 | 0.00 | 0.35 |
| 44 | 1.00 | 0.50 |
| 45 | 0.23 | 0.70 |
| 46 | 0.46 | 0.05 |
| 47 | 0.31 | 0.70 |
| 48 | 0.08 | 0.40 |
| 49 | 0.23 | 0.60 |
| 50 | 0.08 | 0.45 |
| 51 | 0.00 | 0.35 |
| 52 | 0.08 | 0.40 |
| 53 | 0.23 | 0.55 |
| 54 | 0.00 | 0.35 |
| 55 | 0.23 | 0.55 |
| 56 | 0.92 | 0.60 |
| 57 | 0.69 | 0.35 |
| 58 | 0.23 | 0.65 |
| 59 | 0.15 | 0.50 |
| 60 | 0.15 | 0.60 |
| 61 | 0.31 | 0.65 |
| 62 | 0.23 | 0.70 |
| 63 | 0.62 | 0.20 |
| 64 | 0.08 | 0.40 |
| 65 | 0.46 | 0.15 |
| 66 | 0.00 | 0.40 |
| 67 | 0.15 | 0.65 |
| 68 | 0.46 | 0.15 |
| 69 | 0.00 | 0.30 |
| 70 | 0.08 | 0.60 |
| 71 | 0.00 | 0.35 |
| 72 | 0.00 | 0.35 |
| 73 | 0.08 | 0.45 |
| 74 | 0.00 | 0.30 |
| 75 | 0.08 | 0.45 |
| 76 | 0.08 | 0.40 |
| 77 | 0.08 | 0.60 |
| 78 | 0.54 | 0.15 |
| 79 | 0.00 | 0.30 |
| 80 | 0.00 | 0.35 |
| 81 | 0.23 | 0.55 |
| 82 | 0.38 | 1.00 |
| 83 | 0.31 | 0.65 |
| 84 | 0.00 | 0.30 |
| 85 | 0.31 | 0.70 |
| 86 | 0.31 | 0.80 |
| 87 | 0.00 | 0.50 |
| 88 | 0.23 | 0.55 |
| 89 | 0.38 | 0.70 |
| 90 | 0.00 | 0.30 |
| 91 | 0.00 | 0.35 |
| 92 | 0.08 | 0.75 |
| 93 | 0.00 | 0.30 |
| 94 | 0.62 | 0.25 |
| 95 | 0.15 | 0.50 |
| 96 | 0.38 | 0.75 |
| 97 | 0.08 | 0.45 |
| 98 | 0.15 | 0.55 |
| 99 | 0.15 | 0.60 |
| 100 | 0.46 | 0.85 |
| 101 | 0.08 | 0.45 |
| 102 | 0.15 | 0.55 |
| 103 | 0.08 | 0.40 |
| 104 | 0.69 | 1.00 |
| 105 | 0.54 | 0.90 |
| 106 | 0.54 | 0.90 |
| 107 | 0.00 | 0.30 |
| 108 | 0.69 | 1.00 |
| 109 | 0.46 | 0.15 |
| 110 | 0.54 | 0.05 |
| 111 | 0.38 | 0.05 |
| 112 | 0.15 | 0.50 |
| 113 | 0.46 | 0.80 |
| 114 | 0.00 | 0.35 |
| 115 | 0.46 | 0.90 |
| 116 | 0.54 | 0.90 |
| 117 | 0.62 | 0.95 |
| 118 | 0.62 | 1.00 |
| 119 | 0.00 | 0.35 |
| 120 | 0.38 | 0.95 |
| 121 | 0.08 | 0.40 |
| 122 | 0.31 | 0.00 |
| 123 | 0.46 | 0.80 |
| 124 | 0.08 | 0.40 |
| 125 | 0.08 | 0.45 |
| 126 | 0.00 | 0.40 |
| 127 | 0.92 | 0.60 |
| 128 | 0.00 | 0.45 |
| 129 | 0.62 | 1.00 |
| 130 | 0.00 | 0.35 |
| 131 | 0.00 | 0.35 |
| 132 | 0.08 | 0.40 |
| 133 | 0.31 | 0.75 |
| 134 | 0.46 | 0.10 |
| 135 | 0.31 | 0.00 |
| 136 | 0.08 | 0.45 |
| 137 | 0.38 | 0.05 |
| 138 | 0.54 | 0.90 |
| 139 | 1.00 | 0.70 |
| 140 | 0.54 | 0.85 |
| 141 | 0.54 | 0.20 |
| 142 | 0.38 | 0.80 |
| 143 | 0.08 | 0.45 |
| 144 | 0.54 | 0.95 |
| 145 | 0.00 | 0.40 |
| 146 | 0.38 | 0.70 |
| 147 | 0.15 | 0.75 |
| 148 | 0.00 | 0.30 |
| 149 | 0.77 | 0.45 |
| 150 | 0.00 | 0.30 |
| 151 | 0.31 | 0.00 |
| 152 | 0.38 | 0.70 |
| 153 | 0.08 | 0.55 |
| 154 | 0.15 | 0.50 |
| 155 | 0.00 | 0.40 |
| 156 | 0.08 | 0.45 |
| 157 | 0.23 | 0.60 |
| 158 | 0.00 | 0.30 |
| 159 | 0.54 | 0.05 |
| 160 | 0.38 | 0.70 |
| 161 | 0.46 | 0.00 |
| 162 | 0.46 | 0.15 |
| 163 | 0.54 | 0.90 |
| 164 | 0.62 | 0.15 |
| 165 | 0.15 | 0.50 |
| 166 | 0.23 | 0.55 |
| 167 | 0.31 | 0.00 |
| 168 | 0.62 | 0.25 |
| 169 | 0.46 | 0.80 |
| 170 | 0.15 | 0.75 |
| 171 | 0.31 | 0.90 |
| 172 | 0.38 | 0.80 |
| 173 | 0.54 | 0.20 |
| 174 | 0.38 | 0.80 |
| 175 | 0.00 | 0.50 |
| 176 | 0.46 | 0.15 |
| 177 | 0.54 | 0.20 |
| 178 | 0.31 | 0.70 |
| 179 | 0.00 | 0.30 |
| 180 | 0.31 | 0.75 |
| 181 | 0.00 | 0.50 |
| 182 | 0.31 | 0.00 |
| 183 | 0.54 | 0.90 |
| 184 | 0.00 | 0.30 |
| 185 | 0.08 | 0.40 |
| 186 | 0.08 | 0.40 |
| 187 | 0.00 | 0.30 |
| 188 | 0.46 | 0.90 |
| 189 | 0.00 | 0.30 |
| 190 | 0.23 | 0.90 |
| 191 | 0.00 | 0.35 |
| 192 | 0.62 | 0.30 |
| 193 | 0.00 | 0.40 |
| 194 | 0.00 | 0.50 |
| 195 | 0.54 | 0.90 |
| 196 | 0.46 | 0.10 |
| 197 | 0.15 | 0.45 |
| 198 | 0.62 | 0.30 |
| 199 | 0.38 | 0.70 |
| 200 | 0.62 | 0.25 |
| 201 | 0.23 | 0.55 |
| 202 | 0.23 | 0.75 |
| 203 | 0.31 | 0.75 |
| 204 | 0.54 | 0.90 |
| 205 | 0.08 | 0.45 |
| 206 | 0.38 | 0.05 |
| 207 | 0.31 | 0.65 |
| 208 | 0.62 | 0.25 |
| 209 | 0.08 | 0.40 |
| 210 | 0.08 | 0.45 |
| 211 | 0.38 | 0.00 |
| 212 | 0.23 | 0.60 |
| 213 | 0.31 | 0.00 |
| 214 | 0.00 | 0.30 |
| 215 | 0.54 | 0.20 |
| 216 | 0.31 | 0.65 |
| 217 | 0.54 | 0.85 |
| 218 | 0.85 | 0.40 |
| 219 | 0.54 | 0.85 |
| 220 | 0.77 | 0.45 |
| 221 | 0.31 | 0.75 |
| 222 | 0.23 | 0.60 |
| 223 | 0.38 | 0.00 |
| 224 | 0.54 | 0.85 |
| 225 | 0.00 | 0.30 |
| 226 | 0.38 | 0.75 |
| 227 | 0.15 | 0.70 |
| 228 | 0.08 | 0.50 |
| 229 | 0.00 | 0.35 |
| 230 | 0.23 | 0.55 |
| 231 | 0.31 | 0.00 |
| 232 | 0.38 | 0.70 |
| 233 | 0.38 | 0.70 |
| 234 | 0.46 | 0.15 |
| 235 | 0.00 | 0.30 |
| 236 | 0.08 | 0.55 |
| 237 | 0.23 | 0.55 |
| 238 | 0.08 | 0.40 |
| 239 | 0.46 | 0.10 |
| 240 | 0.77 | 0.30 |
| 241 | 0.15 | 0.50 |
| 242 | 0.54 | 0.20 |

3. The method according to claim 1, wherein said separating and identifying said polypeptide markers is by an analytical method selected from the group consisting of: capillary electrophoresis, HPLC, gas-phase ion spectrometry, mass spectrometry, and any combinations thereof.

4. The method according to claim 3, wherein mass spectrometry is used for said identifying of said polypeptide markers.
